SEQUENCE LISTING (9 sequences)

SEQ ID NO: 1 - protein, 239 aa, Homo spaiens
MAHAGRTGYDNREIVMKYIHYKLSGRGYEWDAGDVGAAPPGAAPAPGIFSSQPGHTPHPAASRDPVARTSPLGTPAAPGAAAGPALSPVPPVVHLTLRGAGDDFSRRYRRDFAEMSSQLHLTPFTARGRFATVVEELFRDGVNWGRIVAFFEFGGVMCVESVNREMSPLVDNIALWMTEYLNRHLHTWIGDNGGWDAFVELYGPSMRPLFDFSWLSLKTLLSLALVGACITLGAYLGHK

SEQ ID NO: 2 - DNA, 6492 bp, Homo sapiens
tttctgtgaagcagaagtctgggaatcgatctggaaatcctcctaatttttactccctctccccgcgactcctgattcattgggaagtttcaaatcagctataactggagagtgctgaagattgatgggatcgttgccttatgcatttgttttggttttacaaaaaggaaacttgacagaggatcatgctgtacttaaaaaatacaacatcacagaggaagtagactgatattaacaatacttactaataataacgtgcctcatgaaataaagatccgaaaggaattggaataaaaatttcctgcatctcatgccaagggggaaacaccagaatcaagtgttccgcgtgattgaagacaccccctcgtccaagaatgcaaagcacatccaataaaatagctggattataactcctcttctttctctgggggccgtggggtgggagctggggcgagaggtgccgttggcccccgttgcttttcctctgggaaggatggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtgaagtcaacatgcctgccccaaacaaatatgcaaaaggttcactaaagcagtagaaataatatgcattgtcagtgatgtaccatgaaacaaagctgcaggctgtttaagaaaaaataacacacatataaacatcacacacacagacagacacacacacacacaacaattaacagtcttcaggcaaaacgtcgaatcagctatttactgccaaagggaaatatcatttattttttacattattaagaaaaaaagatttatttatttaagacagtcccatcaaaactcctgtctttggaaatccgaccactaattgccaagcaccgcttcgtgtggctccacctggatgttctgtgcctgtaaacatagattcgctttccatgttgttggccggatcaccatctgaagagcagacggatggaaaaaggacctgatcattggggaagctggctttctggctgctggaggctggggagaaggtgttcattcacttgcatttctttgccctgggggctgtgatattaacagagggagggttcctgtggggggaagtccatgcctccctggcctgaagaagagactctttgcatatgactcacatgatgcatacctggtgggaggaaaagagttgggaacttcagatggacctagtacccactgagatttccacgccgaaggacagcgatgggaaaaatgcccttaaatcataggaaagtatttttttaagctaccaattgtgccgagaaaagcattttagcaatttatacaatatcatccagtaccttaagccctgattgtgtatattcatatattttggatacgcaccccccaactcccaatactggctctgtctgagtaagaaacagaatcctctggaacttgaggaagtgaacatttcggtgacttccgcatcaggaaggctagagttacccagagcatcaggccgccacaagtgcctgcttttaggagaccgaagtccgcagaacctgcctgtgtcccagcttggaggcctggtcctggaactgagccggggccctcactggcctcctccagggatgatcaacagggcagtgtggtctccgaatgtctggaagctgatggagctcagaattccactgtcaagaaagagcagtagaggggtgtggctgggcctgtcaccctggggccctccaggtaggcccgttttcacgtggagcatgggagccacgacccttcttaagacatgtatcactgtagagggaaggaacagaggccctgggcccttcctatcagaaggacatggtgaaggctgggaacgtgaggagaggcaatggccacggcccattttggctgtagcacatggcacgttggctgtgtggccttggcccacctgtgagtttaaagcaaggctttaaatgactttggagagggtcacaaatcctaaaagaagcattgaagtgaggtgtcatggattaattgacccctgtctatggaattacatgtaaaacattatcttgtcactgtagtttggttttatttgaaaacctgacaaaaaaaaagttccaggtgtggaatatgggggttatctgtacatcctggggcattaaaaaaaaaatcaatggtggggaactataaagaagtaacaaaagaagtgacatcttcagcaaataaactaggaaatttttttttcttccagtttagaatcagccttgaaacattgatggaataactctgtggcattattgcattatataccatttatctgtattaactttggaatgtactctgttcaatgtttaatgctgtggttgatatttcgaaagctgctttaaaaaaatacatgcatctcagcgtttttttgtttttaattgtatttagttatggcctatacactatttgtgagcaaaggtgatcgttttctgtttgagatttttatctcttgattcttcaaaagcattctgagaaggtgagataagccctgagtctcagctacctaagaaaaacctggatgtcactggccactgaggagctttgtttcaaccaagtcatgtgcatttccacgtcaacagaattgtttattgtgacagttatatctgttgtccctttgaccttgtttcttgaaggtttcctcgtccctgggcaattccgcatttaattcatggtattcaggattacatgcatgtttggttaaacccatgagattcattcagttaaaaatccagatggcaaatgaccagcagattcaaatctatggtggtttgacctttagagagttgctttacgtggcctgtttcaacacagacccacccagagccctcctgccctccttccgcgggggctttctcatggctgtccttcagggtcttcctgaaatgcagtggtgcttacgctccaccaagaaagcaggaaacctgtggtatgaagccagacctccccggcgggcctcagggaacagaatgatcagacctttgaatgattctaatttttaagcaaaatattattttatgaaaggtttacattgtcaaagtgatgaatatggaatatccaatcctgtgctgctatcctgccaaaatcattttaatggagtcagtttgcagtatgctccacgtggtaagatcctccaagctgctttagaagtaacaatgaagaacgtggacgtttttaatataaagcctgttttgtcttttgttgttgttcaaacgggattcacagagtatttgaaaaatgtatatatattaagaggtcacgggggctaattgctggctggctgccttttgctgtggggttttgttacctggttttaataacagtaaatgtgcccagcctcttggccccagaactgtacagtattgtggctgcacttgctctaagagtagttgatgttgcattttccttattgttaaaaacatgttagaagcaatgaatgtatataaaagcctcaactagtcatttttttctcctcttcttttttttcattatatctaattattttgcagttgggcaacagagaaccatccctattttgtattgaagagggattcacatctgcatcttaactgctctttatgaatgaaaaaacagtcctctgtatgtactcctctttacactggccagggtcagagttaaatagagtatatgcactttccaaattggggacaagggctctaaaaaaagccccaaaaggagaagaacatctgagaacctcctcggccctcccagtccctcgctgcacaaatactccgcaagagaggccagaatgacagctgacagggtctatggccatcgggtcgtctccgaagatttggcaggggcagaaaactctggcaggcttaagatttggaataaagtcacagaattaaggaagcacctcaatttagttcaaacaagacgccaacattctctccacagctcacttacctctctgtgttcagatgtggccttccatttatatgtgatctttgttttattagtaaatgcttatcatctaaagatgtagctctggcccagtgggaaaaattaggaagtgattataaatcgagaggagttataataatcaagattaaatgtaaataatcagggcaatcccaacacatgtctagctttcacctccaggatctattgagtgaacagaattgcaaatagtctctatttgtaattgaacttatcctaaaacaaatagtttataaatgtgaacttaaactctaattaattccaactgtacttttaaggcagtggctgtttttagactttcttatcacttatagttagtaatgtacacctactctatcagagaaaaacaggaaaggctcgaaatacaagccattctaaggaaattagggagtcagttgaaattctattctgatcttattctgtggtgtcttttgcagcccagacaaatgtggttacacactttttaagaaatacaattctacattgtcaagcttatgaaggttccaatcagatctttattgttattcaatttggatctttcagggattttttttttaaattattatgggacaaaggacatttgttggaggggtgggagggaggaagaatttttaaatgtaaaacattcccaagtttggatcagggagttggaagttttcagaataaccagaactaagggtatgaaggacctgtattggggtcgatgtgatgcctctgcgaagaaccttgtgtgacaaatgagaaacattttgaagtttgtggtacgacctttagattccagagacatcagcatggctcaaagtgcagctccgtttggcagtgcaatggtataaatttcaagctggatatgtctaatgggtatttaaacaataaatgtgcagttttaactaacaggatatttaatgacaaccttctggttggtagggacatctgtttctaaatgtttattatgtacaatacagaaaaaaattttataaaattaagcaatgtgaaactgaattggagagtgataatacaagtcctttagtcttacccagtgaatcattctgttccatgtctttggacaaccatgaccttggacaatcatgaaatatgcatctcactggatgcaaagaaaatcagatggagcatgaatggtactgtaccggttcatctggactgccccagaaaaataacttcaagcaaacatcctatcaacaacaaggttgttctgcataccaagctgagcacagaagatgggaacactggtggaggatggaaaggctcgctcaatcaagaaaattctgagactattaataaataagactgtagtgtagatactgagtaaatccatgcacctaaaccttttggaaaatctgccgtgggccctccagatagctcatttcattaagtttttccctccaaggtagaatttgcaagagtgacagtggattgcatttcttttggggaagctttcttttggtggttttgtttattataccttcttaagttttcaaccaaggtttgcttttgttttgagttactggggttatttttgttttaaataaaaataagtgtacaataagtgtttttgtattgaaagcttttgttatcaagattttcatacttttaccttccatggctctttttaagattgatacttttaagaggtggctgatattctgcaacactgtacacataaaaaatacggtaaggatactttacatggttaaggtaaagtaagtctccagttggccaccattagctataatggcactttgtttgtgttgttggaaaaagtcacattgccattaaactttccttgtctgtctagttaatattgtgaagaaaaataaagtacagtgtgagatactg

SEQ ID NO: 3 - protein, 233 aa, Homo sapiens
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 4 - DNA, 2575 bp, Homo sapiens
ggaggaggaagcaagcgagggggctggttcctgagcttcgcaattcctgtgtcgccttctgggctcccagcctgccgggtcgcatgatccctccggccggagctggtttttttgccagccaccgcgaggccggctgagttaccggcatccccgcagccacctcctctcccgacctgtgatacaaaagatcttccgggggctgcacctgcctgcctttgcctaaggcggatttgaatctctttctctcccttcagaatcttatcttggctttggatcttagaagagaatcactaaccagagacgagactcagtgagtgagcaggtgttttggacaatggactggttgagcccatccctattataaaaatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaccagacactgaccatccactctaccctcccacccccttctctgctccaccacatcctccgtccagccgccattgccaccaggagaaccactacatgcagcccatgcccacctgcccatcacagggttgggcccagatctggtcccttgcagctagttttctagaatttatcacacttctgtgagacccccacacctcagttcccttggcctcagaattcacaaaatttccacaaaatctgtccaaaggaggctggcaggtatggaagggtttgtggctgggggcaggagggccctacctgattggtgcaacccttaccccttagcctccctgaaaatgtttttctgccagggagcttgaaagttttcagaacctcttccccagaaaggagactagattgcctttgttttgatgtttgtggcctcagaattgatcattttccccccactctccccacactaacctgggttccctttccttccatccctaccccctaagagccatttaggggccacttttgactagggattcaggctgcttgggataaagatgcaaggaccaggactccctcctcacctctggactggctagagtcctcactcccagtccaaatgtcctccagaagcctctggctagaggccagccccacccaggagggagggggctatagctacaggaagcaccccatgccaaagctagggtggcccttgcagttcagcaccaccctagtcccttcccctccctggctcccatgaccatactgagggaccaactgggcccaagacagatgccccagagctgtttatggcctcagctgcctcacttcctacaagagcagcctgtggcatctttgccttgggctgctcctcatggtgggttcaggggactcagccctgaggtgaaagggagctatcaggaacagctatgggagccccagggtcttccctacctcaggcaggaagggcaggaaggagagcctgctgcatggggtggggtagggctgactagaagggccagtcctgcctggccaggcagatctgtgccccatgcctgtccagcctgggcagccaggctgccaaggccagagtggcctggccaggagctcttcaggcctccctctctcttctgctccacccttggcctgtctcatccccaggggtcccagccaccccgggctctctgctgtacatatttgagactagtttttattccttgtgaagatgatatactatttttgttaagcgtgtctgtatttatgtgtgaggagctgctggcttgcagtgcgcgtgcacgtggagagctggtgcccggagattggacggcctgatgctccctcccctgccctggtccagggaagctggccgagggtcctggctcctgaggggcatctgcccctcccccaacccccaccccacacttgttccagctctttgaaatagtctgtgtgaaggtgaaagtgcagttcagtaataaactgtgtttactcagtgaaaaaaaaaaaaaaaaaa

SEQ ID NO: 5 - protein, 1280 aa, Homo sapiens
MDLEGDRNGGAKKKNFFKLNNKSEKDKKEKKPTVSVFSMFRYSNWLDKLYMVVGTLAAIIHGAGLPLMMLVFGEMTDIFANAGNLEDLMSNITNRSDINDTGFFMNLEEDMTRYAYYYSGIGAGVLVAAYIQVSFWCLAAGRQIHKIRKQFFHAIMRQEIGWFDVHDVGELNTRLTDDVSKINEGIGDKIGMFFQSMATFFTGFIVGFTRGWKLTLVILAISPVLGLSAAVWAKILSSFTDKELLAYAKAGAVAEEVLAAIRTVIAFGGQKKELERYNKNLEEAKRIGIKKAITANISIGAAFLLIYASYALAFWYGTTLVLSGEYSIGQVLTVFFSVLIGAFSVGQASPSIEAFANARGAAYEIFKIIDNKPSIDSYSKSGHKPDNIKGNLEFRNVHFSYPSRKEVKILKGLNLKVQSGQTVALVGNSGCGKSTTVQLMQRLYDPTEGMVSVDGQDIRTINVRFLREIIGVVSQEPVLFATTIAENIRYGRENVTMDEIEKAVKEANAYDFIMKLPHKFDTLVGERGAQLSGGQKQRIAIARALVRNPKILLLDEATSALDTESEAVVQVALDKARKGRTTIVIAHRLSTVRNADVIAGFDDGVIVEKGNHDELMKEKGIYFKLVTMQTAGNEVELENAADESKSEIDALEMSSNDSRSSLIRKRSTRRSVRGSQAQDRKLSTKEALDESIPPVSFWRIMKLNLTEWPYFVVGVFCAIINGGLQPAFAIIFSKIIGVFTRIDDPETKRQNSNLFSLLFLALGIISFITFFLQGFTFGKAGEILTKRLRYMVFRSMLRQDVSWFDDPKNTTGALTTRLANDAAQVKGAIGSRLAVITQNIANLGTGIIISFIYGWQLTLLLLAIVPIIAIAGVVEMKMLSGQALKDKKELEGSGKIATEAIENFRTVVSLTQEQKFEHMYAQSLQVPYRNSLRKAHIFGITFSFTQAMMYFSYAGCFRFGAYLVAHKLMSFEDVLLVFSAVVFGAMAVGQVSSFAPDYAKAKISAAHIIMIIEKTPLIDSYSTEGLMPNTLEGNVTFGEVVFNYPTRPDIPVLQGLSLEVKKGQTLALVGSSGCGKSTVVQLLERFYDPLAGKVLLDGKEIKRLNVQWLRAHLGIVSQEPILFDCSIAENIAYGDNSRVVSQEEIVRAAKEANIHAFIESLPNKYSTKVGDKGTQLSGGQKQRIAIARALVRQPHILLLDEATSALDTESEKVVQEALDKAREGRTCIVIAHRLSTIQNADLIVVFQNGRVKEHGTHQQLLAQKGIYFSMVSVQAGTKRQ

SEQ ID NO: 6 - DNA, 4872 bp, Homo sapiens
tattcagatattctccagattcctaaagattagagatcatttctcattctcctaggagtactcacttcaggaagcaaccagataaaagagaggtgcaacggaagccagaacattcctcctggaaattcaacctgtttcgcagtttctcgaggaatcagcattcagtcaatccgggccgggagcagtcatctgtggtgaggctgattggctgggcaggaacagcgccggggcgtgggctgagcacagccgcttcgctctctttgccacaggaagcctgagctcattcgagtagcggctcttccaagctcaaagaagcagaggccgctgttcgtttcctttaggtctttccactaaagtcggagtatcttcttccaaaatttcacgtcttggtggccgttccaaggagcgcgaggtcggaatggatcttgaaggggaccgcaatggaggagcaaagaagaagaacttttttaaactgaacaataaaagtgaaaaagataagaaggaaaagaaaccaactgtcagtgtattttcaatgtttcgctattcaaattggcttgacaagttgtatatggtggtgggaactttggctgccatcatccatggggctggacttcctctcatgatgctggtgtttggagaaatgacagatatctttgcaaatgcaggaaatttagaagatctgatgtcaaacatcactaatagaagtgatatcaatgatacagggttcttcatgaatctggaggaagacatgaccaggtatgcctattattacagtggaattggtgctggggtgctggttgctgcttacattcaggtttcattttggtgcctggcagctggaagacaaatacacaaaattagaaaacagttttttcatgctataatgcgacaggagataggctggtttgatgtgcacgatgttggggagcttaacacccgacttacagatgatgtctccaagattaatgaaggaattggtgacaaaattggaatgttctttcagtcaatggcaacatttttcactgggtttatagtaggatttacacgtggttggaagctaacccttgtgattttggccatcagtcctgttcttggactgtcagctgctgtctgggcaaagatactatcttcatttactgataaagaactcttagcgtatgcaaaagctggagcagtagctgaagaggtcttggcagcaattagaactgtgattgcatttggaggacaaaagaaagaacttgaaaggtacaacaaaaatttagaagaagctaaaagaattgggataaagaaagctattacagccaatatttctataggtgctgctttcctgctgatctatgcatcttatgctctggccttctggtatgggaccaccttggtcctctcaggggaatattctattggacaagtactcactgtattcttttctgtattaattggggcttttagtgttggacaggcatctccaagcattgaagcatttgcaaatgcaagaggagcagcttatgaaatcttcaagataattgataataagccaagtattgacagctattcgaagagtgggcacaaaccagataatattaagggaaatttggaattcagaaatgttcacttcagttacccatctcgaaaagaagttaagatcttgaagggtctgaacctgaaggtgcagagtgggcagacggtggccctggttggaaacagtggctgtgggaagagcacaacagtccagctgatgcagaggctctatgaccccacagaggggatggtcagtgttgatggacaggatattaggaccataaatgtaaggtttctacgggaaatcattggtgtggtgagtcaggaacctgtattgtttgccaccacgatagctgaaaacattcgctatggccgtgaaaatgtcaccatggatgagattgagaaagctgtcaaggaagccaatgcctatgactttatcatgaaactgcctcataaatttgacaccctggttggagagagaggggcccagttgagtggtgggcagaagcagaggatcgccattgcacgtgccctggttcgcaaccccaagatcctcctgctggatgaggccacgtcagccttggacacagaaagcgaagcagtggttcaggtggctctggataaggccagaaaaggtcggaccaccattgtgatagctcatcgtttgtctacagttcgtaatgctgacgtcatcgctggtttcgatgatggagtcattgtggagaaaggaaatcatgatgaactcatgaaagagaaaggcatttacttcaaacttgtcacaatgcagacagcaggaaatgaagttgaattagaaaatgcagctgatgaatccaaaagtgaaattgatgccttggaaatgtcttcaaatgattcaagatccagtctaataagaaaaagatcaactcgtaggagtgtccgtggatcacaagcccaagacagaaagcttagtaccaaagaggctctggatgaaagtatacctccagtttccttttggaggattatgaagctaaatttaactgaatggccttattttgttgttggtgtattttgtgccattataaatggaggcctgcaaccagcatttgcaataatattttcaaagattataggggtttttacaagaattgatgatcctgaaacaaaacgacagaatagtaacttgttttcactattgtttctagcccttggaattatttcttttattacatttttccttcagggtttcacatttggcaaagctggagagatcctcaccaagcggctccgatacatggttttccgatccatgctcagacaggatgtgagttggtttgatgaccctaaaaacaccactggagcattgactaccaggctcgccaatgatgctgctcaagttaaaggggctataggttccaggcttgctgtaattacccagaatatagcaaatcttgggacaggaataattatatccttcatctatggttggcaactaacactgttactcttagcaattgtacccatcattgcaatagcaggagttgttgaaatgaaaatgttgtctggacaagcactgaaagataagaaagaactagaaggttctgggaagatcgctactgaagcaatagaaaacttccgaaccgttgtttctttgactcaggagcagaagtttgaacatatgtatgctcagagtttgcaggtaccatacagaaactctttgaggaaagcacacatctttggaattacattttccttcacccaggcaatgatgtatttttcctatgctggatgtttccggtttggagcctacttggtggcacataaactcatgagctttgaggatgttctgttagtattttcagctgttgtctttggtgccatggccgtggggcaagtcagttcatttgctcctgactatgccaaagccaaaatatcagcagcccacatcatcatgatcattgaaaaaacccctttgattgacagctacagcacggaaggcctaatgccgaacacattggaaggaaatgtcacatttggtgaagttgtattcaactatcccacccgaccggacatcccagtgcttcagggactgagcctggaggtgaagaagggccagacgctggctctggtgggcagcagtggctgtgggaagagcacagtggtccagctcctggagcggttctacgaccccttggcagggaaagtgctgcttgatggcaaagaaataaagcgactgaatgttcagtggctccgagcacacctgggcatcgtgtcccaggagcccatcctgtttgactgcagcattgctgagaacattgcctatggagacaacagccgggtggtgtcacaggaagagattgtgagggcagcaaaggaggccaacatacatgccttcatcgagtcactgcctaataaatatagcactaaagtaggagacaaaggaactcagctctctggtggccagaaacaacgcattgccatagctcgtgcccttgttagacagcctcatattttgcttttggatgaagccacgtcagctctggatacagaaagtgaaaaggttgtccaagaagccctggacaaagccagagaaggccgcacctgcattgtgattgctcaccgcctgtccaccatccagaatgcagacttaatagtggtgtttcagaatggcagagtcaaggagcatggcacgcatcagcagctgctggcacagaaaggcatctatttttcaatggtcagtgtccaggctggaacaaagcgccagtgaactctgactgtatgagatgttaaatactttttaatatttgtttagatatgacatttattcaaagttaaaagcaaacacttacagaattatgaagaggtatctgtttaacatttcctcagtcaagttcagagtcttcagagacttcgtaattaaaggaacagagtgagagacatcatcaagtggagagaaatcatagtttaaactgcattataaattttataacagaattaaagtagattttaaaagataaaatgtgtaattttgtttatattttcccatttggactgtaactgactgccttgctaaaagattatagaagtagcaaaaagtattgaaatgtttgcataaagtgtctataataaaactaaactttcatgtgactggagtcatcttgtccaaactgcctgtgaatatatcttctctcaattggaatattgtagataacttctgctttaaaaaagttttctttaaatatacctactcatttttgtgggaatggttaagcagtttaaataattcctgttgtatatgtctattcacattgggtcttacagaaccatctggcttcattcttcttggacttgatcctgctgattcttgcatttccacat

SEQ ID NO: 7 - protein, 4 aa, Artificial Sequence
  synthetic proteasome inhibitor
XITX

SEQ ID NO: 8 - protein, 4 aa, Artificial Sequence
  Synthetic proteasome inhibitor
XXAX

SEQ ID NO: 9 - protein, 4 aa, Artificial Sequence
  Synthetic proteasome inhibitor
TLLX

(19) United States
(12) Patent Application Publication
(10) Pub. No.: US 2011/0269699 A1
(43) Pub. Date: Nov. 3, 2011

(54) CANCER THERAPY

(76) Inventors: Mitchell Keegan, Berlin, MA (US); Ricky W. Johnstone, Fairfield (AU); Andrea Newbold, Port Melbourne (AU); Leonie Cluse, Thornbury (AU)

(21) Appl. No.: 13/124,838

(22) PCT Filed: Oct. 24, 2008
(86) PCT No.: PCT/US08/81107

§ 371 Date: Jul. 12, 2011

(51) Int. Cl. A61K 38/15; A61P 35/00; C12Q 1/68; C12N 5/09; C12Q 1/02

(52) U.S. Cl. 514/19.9; 435/375; 435/29; 435/6.1

(57) ABSTRACT

The invention provides therapy for treating cancers, such as Bcl-2⁺ cancers, and Bcl-X_{L}⁻ cancers, and other neoplasms, using romidepsin. The invention provides, inter alia, methods of treating lymphomas, e.g., lymphomas characterized by one or more of Bcl-2 expression, lack of overexpression of Bcl-X_{L}, lack of overexpression of P-glycoprotein, with romidepsin. In some embodiments, the lymphoma is a cutaneous T cell lymphoma. In some embodiments, the lymphoma is a peripheral T cell lymphoma. Romidepsin can be administered a dosages ranging from 0.5 mg/m² to approximately 28 mg/m² (e.g., from 1 mg/m² to 15 mg/m², from 4 mg/m² to 15 mg/m², from 8 mg/m² to 14 mg/m², or from 4 mg/m² to approximately 10 mg/m²). Romidepsin can be administered with a second agent, such as a cytotoxic agent, a steroidal agent, a proteasome inhibitor, or a kinase inhibitor.

### BACKGROUND OF THE INVENTION

Romidepsin is a natural product which was isolated from Chromobacterium violaceum by Fujisawa Pharmaceuticals. See Published Japanese Patent Application Hei 7 (1995)-64872; U.S. Pat. No. 4,977,138, issued Dec. 11, 1990, which is incorporated herein by reference. It is a bicyclic peptide consisting of four amino acid residues (D-valine, D-cysteine, dehydrobutyrine, and L-valine) and a novel acid (3-hydroxy-7-mercapto-4-heptenoic acid). Romidepsin is a depsipeptide which contains both amide and ester bonds. In addition to fermentation from C. violaceum, romidepsin can also be prepared by synthetic or semi-synthetic means. The total synthesis of romidepsin reported by Kahn et al. involves 14 steps and yields romidepsin in 18% overall yield. J. Am. Chem. Soc. 118:7237-7238, 1996. The structure of romidepsin is shown below:

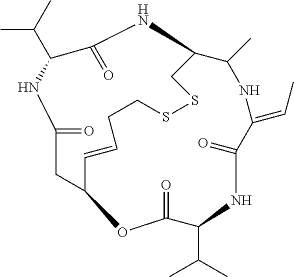

Romidepsin has been shown to have anti-microbial, immunosuppressive, and anti-tumor activities. It is thought to act by selectively inhibiting deacetylases (e.g., histone deacetylase (HDAC), tubulin deacetylase (TDAC)), promising new targets for the development of anti-cancer therapies. Nakajima et al., Experimental Cell Res. 241:126-133, 1998. One mode of action is thought to involve the inhibition of one or more classes of histone deacetylases (HDAC).

Histone deacetylase is a metallodeacetylation enzyme having zinc in its active site. Finnin et al., Nature, 401:188-193, 1999. This enzyme is thought to regulate gene expression by enhancing the acetylation of histones, thereby inducing chromatin relaxation and generally, but not universally, transcriptional activation. Although these enzymes are known as HDACs, they have also been implicated in various other cellular processes. For example, HDAC inhibitors have been found to trigger apoptosis in tumor cells through diverse mechanisms, including the up-regulation of death receptors, Bid cleavage, ROS generation, Hsp90 dysregulation, and ceramide generation, among others. Several HDAC inhibitors have entered the clinical arena and are demonstrating activity in both hematologic and non-hematologic malignancies. Romidepsin has shown impressive activity in certain hematologic malignancies, particularly T-cell lymphoma (Piekarz et al. “A review of depsipeptide and other histone deacetylase inhibitors in clinical trials” Curr. Pharm. Des. 10:2289-98, 2004; incorporated herein by reference).

In addition to romidepsin, various derivatives have been prepared and studied. The following patents and patent applications describe various derivatives of romidepsin: U.S. Pat. No. 6,548,479; WO 05/0209134; WO 05/058298; and WO 06/129105; each of which is incorporated herein by reference.

### SUMMARY OF THE INVENTION

It has been discovered that an HDAC inhibitor, romidepsin, is effective in inducing apoptosis of cancer cells that express the anti-apoptotic factor, Bcl-2. The invention provides novel methods for evaluating Bcl-2 expression and expression of other factors such as Bcl-X_{L} and P-glycoprotein, for treating cancers with romidepsin and for identifying subjects for treatment. Accordingly, methods of treating cancers (e.g., lymphomas) with romidepsin, based on expression of particular factors, are disclosed herein. The invention also provides methods of treating cells that express particular factors (e.g., in vitro methods) by administering romidepsin. These methods stem from the recognition that romidepsin is effective in inducing apoptosis of cancers that overexpress Bcl-2, such as lymphomas (e.g., cutaneous T cell lymphoma), and that romidepsin provides a therapeutic benefit for treating such cancers when administered in vivo. Romidepsin treatment can be particularly beneficial for treatment of Bcl-2⁺ cancers that do not overexpress Bcl-X_{L} or P-glycoprotein.

In one aspect, the invention provides a method of treating a lymphoma in a subject (e.g., a human) by providing a subject identified as having a lymphoma that expresses Bcl-2 (e.g., a lymphoma that overexpresses Bcl-2), and administering a therapeutically effective amount of romidepsin to the subject. In some embodiments, expression of Bcl-2 in cells of the lymphoma is at least 10%, 25%, 50%, 100%, 200%, 300%, 400%, or 500% greater than expression of Bcl-2 in normal, non-cancerous cells of the same cell type as the lymphoma. In certain embodiments, the method includes a step wherein the subject is identified as having a lymphoma that expresses Bcl-2. Thus, the method can include determining Bcl-2 expression in cells of the lymphoma. In some embodiments, Bcl-2 expression (e.g., Bcl-2 polypeptide expression, and/or Bcl-2 mRNA expression) is determined in vitro in a sample from the lymphoma. Bcl-2 expression can be determined, e.g., by PCR (e.g., RT-PCR, quantitative RT-PCR), in situ hybridization (e.g., fluorescence in situ hybridization), microarray analysis, Northern blot, immunoassays (e.g., Western blot, FACS, immunohistochemistry), and other methods. In some embodiments, cells of the lymphoma have a chromosomal translocation of a Bcl-2 gene that results in Bcl-2 overexpression. In some embodiments, cells of the lymphoma do not have a chromosomal translocation of a Bcl-2 gene (e.g., Bcl-2 overexpression in the cells is due to a mechanism other than Bcl-2 translocation). In some embodiments, the subject is administered a higher dose of romidepsin than a dose that is administered to a subject having a lymphoma that does not express Bcl-2.

In some embodiments, the lymphoma does not overexpress Bcl-X_{L}. In some embodiments, the lymphoma does not express Bcl-X_{L}. In certain embodiments, the lymphoma overexpresses Bcl-2 but does not overexpress Bcl-X_{L}. In some embodiments, expression of Bcl-2 is equal to or greater than expression of Bcl-X_{L} in cells of the lymphoma (e.g., expression of Bcl-2 is at least 25%, 50%, 100%, 150%, or 200% greater than expression of Bcl-X_{L}). The method can include determining Bcl-X_{L} expression in cells of the lymphoma (e.g., wherein Bcl-X_{L} polypeptide and/or mRNA expression is determined in vitro in a sample from the lymphoma). Bcl-X_{L} expression can be determined, e.g., by PCR (e.g., RT-PCR, quantitative RT-PCR), in situ hybridization (e.g., fluorescence in situ hybridization), microarray analysis, Northern blot, immunoassays (e.g., Western blot, FACS, immunohistochemistry), and other methods.

In some embodiments, the lymphoma does not overexpress P-glycoprotein. The method can include determining P-glycoprotein expression in cells of the lymphoma.

In some embodiments, the lymphoma is a T cell lymphoma (e.g., a cutaneous T cell lymphoma (CTCL), or a peripheral T cell lymphoma (PTCL)). In some embodiments, the lymphoma is a non-Hodgkin's lymphoma. In other embodiments, the lymphoma is a Hodgkin's lymphoma. In some embodiments, the lymphoma is a follicular lymphoma, a B cell lymphoma, a diffuse large B cell lymphoma, a mantle cell lymphoma, or a Burkitt's lymphoma.

In some embodiments, the lymphoma is a refractory lymphoma (e.g., a lymphoma that is refractory to chemotherapy). In some embodiments, the lymphoma is a relapsed lymphoma. In some embodiments, the lymphoma is a steroid-resistant lymphoma.

In certain embodiments, romidepsin is administered at a dosage that ranges from approximately 0.5 mg/m² to approximately 28 mg/m² (e.g., from approximately 4 mg/m² to approximately 10 mg/m²). In certain embodiments, romidepsin is administered intravenously. Romidepsin can be administered bimonthly, monthly, triweekly, biweekly, weekly, twice a week, daily, or at variable intervals.

In some embodiments, the method further includes administering a second anti-neoplastic agent, such as an inhibitor of Bcl-X_{L} expression or activity, a proteasome inhibitor, a kinase inhibitor, a nucleoside analog, a mitotic inhibitor, a cytotoxic agent, or a steroidal agent. The second anti-neoplastic agent can be administered together with, prior to, or following the administration of romidepsin.

In another aspect, the invention features a method of treating Bcl-2-expressing lymphoma cells in vitro. The method includes providing lymphoma cells identified as expressing Bcl-2 (e.g., cells that overexpress Bcl-2), and administering romidepsin to the cells. In some embodiments, romidepsin is administered to the cells at a concentration and for a period of time sufficient to kill the cells. In some embodiments, the method includes determining Bcl-2 expression (e.g., Bcl-2 polypeptide expression and/or Bcl-2 mRNA expression) in the cells, prior to administering romidepsin.

In some embodiments, the cells do not overexpress Bcl-X_{L}. In some embodiments, the cells do not express Bcl-X_{L}. In some embodiments, expression of Bcl-2 is equal to or greater than expression of Bcl-X_{L} in the cells (e.g., expression of Bcl-2 is at least 25%, 50%, 100%, 150%, or 200% greater than expression of Bcl-X_{L}). The method can include determining Bcl-X_{L} expression (e.g., Bcl-X_{L} polypeptide expression and/or Bcl-X_{L} mRNA expression) in the cells.

In some embodiments, romidepsin is administered for at least 24 hours (e.g., for at least 72 hours). In some embodiments, romidepsin is administered at a concentration of at least 1 nmol/L (e.g., at least 3 nmol/L).

In another aspect, the invention features a method for identifying a candidate for treatment with romidepsin by providing a sample from a subject having a lymphoma and determining Bcl-2 expression in cells of the lymphoma, wherein expression of Bcl-2 (e.g., overexpression of Bcl-2) in cells of the lymphoma indicates that the subject is a candidate for treatment with romidepsin.

In another aspect, the invention features a method for identifying a candidate lymphoma patient for treatment with romidepsin by providing a sample from a subject having a lymphoma and determining Bcl-2 and Bcl-X_{L} expression in cells of the lymphoma, wherein expression of Bcl-2 which is equal to or greater than expression of Bcl-X_{L} in cells of the lymphoma indicates that the subject is a candidate for treatment with romidepsin.

In a further aspect, the invention features a method for identifying a candidate lymphoma patient for treatment with romidepsin by providing a sample from a subject having a lymphoma, determining Bcl-X_{L} expression in cells of the lymphoma, wherein a lack of overexpression of Bcl-X_{L} in cells of the lymphoma indicates that the subject is a candidate for treatment with romidepsin.

In another aspect, the invention features a method of treating a lymphoma in a subject by providing a subject identified as having a lymphoma that lacks expression of Bcl-X_{L} and administering a therapeutically effective amount of romidepsin to the subject. Methods described above are based, at least in part, on the surprising discovery that romidepsin is effective in inducing apoptosis of cancer cells that express (e.g., overexpress) the anti-apoptotic factor, Bcl-2. The discovery that romidepsin overcomes the anti-apoptotic effects of Bcl-2 indicates that this agent can be used to induce apoptosis of cells in which the expression of other anti- and pro-apoptotic factors is disregulated. Thus, in certain aspects, the invention features methods of treating lymphomas characterized by overexpression of anti-apoptotic factors and/or underexpression of pro-apoptotic factors, which anti- and pro-apoptotic factors are members of a Bcl family or Bcl pathway. Anti-apoptotic factors that are members of the Bcl family include, e.g., Bcl-W, Mcl-1, Bfl-1/A1, BOO/DIVA, and NRH/NR-13. Pro-apoptotic factors that are members of the Bcl family include, e.g., multidomain pro-apoptotic factors such as Bax, Bak, and Bok/Mtd, and BH3-domain only factors such as Bid, Bad, Bik, Blk, Bmf, Bnip3, Hrk, Nix, Noxa, Puma, and Spike. These pro- and anti-apoptotic factors are described, e.g., in Walensky, Cell Death Different. 13:1339-1350, 2006; Aouacheria et al., Oncogene 20(41):5846-55, 2001; and Zamzami et al., Oncogene 16: 2265-2282, 1998). Expression of these factors can be determined according to any method described herein.

The methods can include providing a subject identified as having a lymphoma that expresses one or more Bcl family anti-apoptotic factors, e.g., selected from Bcl-W, Mcl-1, Bfl-1/A1, BOO/DIVA, and NRH/NR-13 (e.g., a lymphoma that overexpresses one or more of the anti-apoptotic factors), and administering a therapeutically effective amount of romidepsin to the subject. The anti-apoptotic factor is a factor other than Bcl-X_{L}. In some embodiments, the method includes a step wherein the subject is identified as having a lymphoma that expresses the anti-apoptotic factor. The method can include determining expression of the anti-apoptotic factor in cells of the lymphoma. In some embodiments, the lymphoma expresses Bcl-2 and one or more anti-apoptotic factors selected from Bcl-W, Mcl-1, Bfl-1/A1, BOO/DIVA, and NRH/NR-13.

The methods can include providing a subject identified as having a lymphoma that underexpresses (e.g., lacks detectable expression of) one or more Bcl family pro-apoptotic factors selected from Bax, Bak, and Bok/Mtd, Bid, Bad, Bik, Blk, Bmf, Bnip3, Hrk, Nix, Noxa, Puma, and Spike, and administering a therapeutically effective amount of romidepsin to the subject. In some embodiments, the method includes a step wherein the subject is identified as having a lymphoma that underexpresses the pro-apoptotic factor. The method can include determining expression of the pro-apoptotic factor in cells of the lymphoma. In some embodiments, the lymphoma expresses Bcl-2 and underexpresses one or more pro-apoptotic factors selected from Bax, Bak, and Bok/Mtd, Bid, Bad, Bik, Blk, Bmf, Bnip3, Hrk, Nix, Noxa, Puma, and Spike.

### Definitions

Definitions of other terms used throughout the specification include:

As used herein and in the appended claims, the singular forms “a”, “an”, and “the” include the plural reference unless the context clearly indicates otherwise. Thus, for example, a reference to “a cell” includes a plurality of such cells.

“Animal”: As used herein, the term “animal” refers to any member of the animal kingdom. In some embodiments, “animal” refers to a human, at any stage of development. In some embodiments, “animal” refers to a non-human animal, at any stage of development. In some embodiments, animals include, but are not limited to, mammals, birds, reptiles, amphibians, fish, and/or worms. In certain embodiments, the non-human animal is a mammal (e.g., a rodent, a mouse, a rat, a rabbit, a monkey, a dog, a cat, a sheep, cattle, a primate, and/or a pig). In some embodiments, an animal may be a transgenic animal, genetically-engineered animal, and/or clone.

“Bcl-2”: As used herein, the term “Bcl-2”, also known as B-cell lymphoma-2, refers to a Bcl-2 polypeptide or the gene encoding the polypeptide. A Bcl-2 polypeptide is a multidomain, integral outer mitochondrial membrane protein that inhibits apoptosis. Nucleotide sequences encoding human Bcl-2 polypeptides are found in GenBank under Acc. Nos. NM_{—}000633.2 and NM_{—}000657.2. Exemplary human Bcl-2 polypeptides sequences are found under Acc. Nos. NP_{—}000624.2, NP_{—}000648.2, and ABX60202.1. A genomic sequence which includes a human Bcl-2 gene sequence is found under Acc. No. NC_{—}000018.8. “Bcl-2”, as used herein, includes human and non-human forms of Bcl-2. Sequences of non-human Bcl-2 genes and polypeptides are known. For example, murine and rat Bcl-2 polypeptide sequence are found under Acc. Nos. NP_{—}033871.2 and NP_{—}058689.1, respectively. The GenBank database sequence entries above are incorporated herein by reference.

An amino acid sequence of a human Bcl-2 polypeptide, found under GenBank Acc. No. NP_{—}000624.2, is as follows:

(SEQ ID NO: 1)
MAHAGRTGYDNREIVMKYIHYKLSQRGYEWDAGDVGAAPPGAAPAPGIFSSQPGHTPHPAASRDPVARTSPLQTP
AAPGAAAGPALSPVPPVVHLTLRQAGDDFSRRYRRDFAEMSSQLHLTPFTARGRFATVVEELFRDGVNWGRIVAF
FEFGGVMCVESVNREMSPLVDNIALWMTEYLNRHLHTWIQDNGGWDAFVELYGPSMRPLFDFSWLSLKTLLSLAL
VGACITLGAYLGHK.

A nucleotide sequence encoding a human Bcl-2 polypeptide, found in GenBank under Acc. No. NM_{—}000633.2, is as follows:

(SEQ ID NO: 2)
TTTCTGTGAAGCAGAAGTCTGGGAATCGATCTGGAAATCCTCCTAATTTTTACTCCCTCTCCCCGCGACTCCTGA
TTCATTGGGAAGTTTCAAATCAGCTATAACTGGAGAGTGCTGAAGATTGATGGGATCGTTGCCTTATGCATTTGT
TTTGGTTTTACAAAAAGGAAACTTGACAGAGGATCATGCTGTACTTAAAAAATACAACATCACAGAGGAAGTAGA
CTGATATTAACAATACTTACTAATAATAACGTGCCTCATGAAATAAAGATCCGAAAGGAATTGGAATAAAAATTT
CCTGCATCTCATGCCAAGGGGGAAACACCAGAATCAAGTGTTCCGCGTGATTGAAGACACCCCCTCGTCCAAGAA
TGCAAAGCACATCCAATAAAATAGCTGGATTATAACTCCTCTTCTTTCTCTGGGGGCCGTGGGGTGGGAGCTGGG
GCGAGAGGTGCCGTTGGCCCCCGTTGCTTTTCCTCTGGGAAGGATGGCGCACGCTGGGAGAACAGGGTACGATAA
CCGGGAGATAGTGATGAAGTACATCCATTATAAGCTGTCGCAGAGGGGCTACGAGTGGGATGCGGGAGATGTGGG
CGCCGCGCCCCCGGGGGCCGCCCCCGCACCGGGCATCTTCTCCTCCCAGCCCGGGCACACGCCCCATCCAGCCGC
ATCCCGGGACCCGGTCGCCAGGACCTCGCCGCTGCAGACCCCGGCTGCCCCCGGCGCCGCCGCGGGGCCTGCGCT
CAGCCCGGTGCCACCTGTGGTCCACCTGACCCTCCGCCAGGCCGGCGACGACTTCTCCCGCCGCTACCGCCGCGA
CTTCGCCGAGATGTCCAGCCAGCTGCACCTGACGCCCTTCACCGCGCGGGGACGCTTTGCCACGGTGGTGGAGGA
GCTCTTCAGGGACGGGGTGAACTGGGGGAGGATTGTGGCCTTCTTTGAGTTCGGTGGGGTCATGTGTGTGGAGAG
CGTCAACCGGGAGATGTCGCCCCTGGTGGACAACATCGCCCTGTGGATGACTGAGTACCTGAACCGGCACCTGCA
CACCTGGATCCAGGATAACGGAGGCTGGGATGCCTTTGTGGAACTGTACGGCCCCAGCATGCGGCCTCTGTTTGA
TTTCTCCTGGCTGTCTCTGAAGACTCTGCTCAGTTTGGCCCTGGTGGGAGCTTGCATCACCCTGGGTGCCTATCT
GGGCCACAAGTGAAGTCAACATGCCTGCCCCAAACAAATATGCAAAAGGTTCACTAAAGCAGTAGAAATAATATG
CATTGTCAGTGATGTACCATGAAACAAAGCTGCAGGCTGTTTAAGAAAAAATAACACACATATAAACATCACACA
CACAGACAGACACACACACACACAACAATTAACAGTCTTCAGGCAAAACGTCGAATCAGCTATTTACTGCCAAAG
GGAAATATCATTTATTTTTTACATTATTAAGAAAAAAAGATTTATTTATTTAAGACAGTCCCATCAAAACTCCTG
TCTTTGGAAATCCGACCACTAATTGCCAAGCACCGCTTCGTGTGGCTCCACCTGGATGTTCTGTGCCTGTAAACA
TAGATTCGCTTTCCATGTTGTTGGCCGGATCACCATCTGAAGAGCAGACGGATGGAAAAAGGACCTGATCATTGG
GGAAGCTGGCTTTCTGGCTGCTGGAGGCTGGGGAGAAGGTGTTCATTCACTTGCATTTCTTTGCCCTGGGGGCTG
TGATATTAACAGAGGGAGGGTTCCTGTGGGGGGAAGTCCATGCCTCCCTGGCCTGAAGAAGAGACTCTTTGCATA
TGACTCACATGATGCATACCTGGTGGGAGGAAAAGAGTTGGGAACTTCAGATGGACCTAGTACCCACTGAGATTT
CCACGCCGAAGGACAGCGATGGGAAAAATGCCCTTAAATCATAGGAAAGTATTTTTTTAAGCTACCAATTGTGCC
GAGAAAAGCATTTTAGCAATTTATACAATATCATCCAGTACCTTAAGCCCTGATTGTGTATATTCATATATTTTG
GATACGCACCCCCCAACTCCCAATACTGGCTCTGTCTGAGTAAGAAACAGAATCCTCTGGAACTTGAGGAAGTGA
ACATTTCGGTGACTTCCGCATCAGGAAGGCTAGAGTTACCCAGAGCATCAGGCCGCCACAAGTGCCTGCTTTTAG
GAGACCGAAGTCCGCAGAACCTGCCTGTGTCCCAGCTTGGAGGCCTGGTCCTGGAACTGAGCCGGGGCCCTCACT
GGCCTCCTCCAGGGATGATCAACAGGGCAGTGTGGTCTCCGAATGTCTGGAAGCTGATGGAGCTCAGAATTCCAC
TGTCAAGAAAGAGCAGTAGAGGGGTGTGGCTGGGCCTGTCACCCTGGGGCCCTCCAGGTAGGCCCGTTTTCACGT
GGAGCATGGGAGCCACGACCCTTCTTAAGACATGTATCACTGTAGAGGGAAGGAACAGAGGCCCTGGGCCCTTCC
TATCAGAAGGACATGGTGAAGGCTGGGAACGTGAGGAGAGGCAATGGCCACGGCCCATTTTGGCTGTAGCACATG
GCACGTTGGCTGTGTGGCCTTGGCCCACCTGTGAGTTTAAAGCAAGGCTTTAAATGACTTTGGAGAGGGTCACAA
ATCCTAAAAGAAGCATTGAAGTGAGGTGTCATGGATTAATTGACCCCTGTCTATGGAATTACATGTAAAACATTA
TCTTGTCACTGTAGTTTGGTTTTATTTGAAAACCTGACAAAAAAAAAGTTCCAGGTGTGGAATATGGGGGTTATC
TGTACATCCTGGGGCATTAAAAAAAAAATCAATGGTGGGGAACTATAAAGAAGTAACAAAAGAAGTGACATCTTC
AGCAAATAAACTAGGAAATTTTTTTTTCTTCCAGTTTAGAATCAGCCTTGAAACATTGATGGAATAACTCTGTGG
CATTATTGCATTATATACCATTTATCTGTATTAACTTTGGAATGTACTCTGTTCAATGTTTAATGCTGTGGTTGA
TATTTCGAAAGCTGCTTTAAAAAAATACATGCATCTCAGCGTTTTTTTGTTTTTAATTGTATTTAGTTATGGCCT
ATACACTATTTGTGAGCAAAGGTGATCGTTTTCTGTTTGAGATTTTTATCTCTTGATTCTTCAAAAGCATTCTGA
GAAGGTGAGATAAGCCCTGAGTCTCAGCTACCTAAGAAAAACCTGGATGTCACTGGCCACTGAGGAGCTTTGTTT
CAACCAAGTCATGTGCATTTCCACGTCAACAGAATTGTTTATTGTGACAGTTATATCTGTTGTCCCTTTGACCTT
GTTTCTTGAAGGTTTCCTCGTCCCTGGGCAATTCCGCATTTAATTCATGGTATTCAGGATTACATGCATGTTTGG
TTAAACCCATGAGATTCATTCAGTTAAAAATCCAGATGGCAAATGACCAGCAGATTCAAATCTATGGTGGTTTGA
CCTTTAGAGAGTTGCTTTACGTGGCCTGTTTCAACACAGACCCACCCAGAGCCCTCCTGCCCTCCTTCCGCGGGG
GCTTTCTCATGGCTGTCCTTCAGGGTCTTCCTGAAATGCAGTGGTGCTTACGCTCCACCAAGAAAGCAGGAAACC
TGTGGTATGAAGCCAGACCTCCCCGGCGGGCCTCAGGGAACAGAATGATCAGACCTTTGAATGATTCTAATTTTT
AAGCAAAATATTATTTTATGAAAGGTTTACATTGTCAAAGTGATGAATATGGAATATCCAATCCTGTGCTGCTAT
CCTGCCAAAATCATTTTAATGGAGTCAGTTTGCAGTATGCTCCACGTGGTAAGATCCTCCAAGCTGCTTTAGAAG
TAACAATGAAGAACGTGGACGTTTTTAATATAAAGCCTGTTTTGTCTTTTGTTGTTGTTCAAACGGGATTCACAG
AGTATTTGAAAAATGTATATATATTAAGAGGTCACGGGGGCTAATTGCTGGCTGGCTGCCTTTTGCTGTGGGGTT
TTGTTACCTGGTTTTAATAACAGTAAATGTGCCCAGCCTCTTGGCCCCAGAACTGTACAGTATTGTGGCTGCACT
TGCTCTAAGAGTAGTTGATGTTGCATTTTCCTTATTGTTAAAAACATGTTAGAAGCAATGAATGTATATAAAAGC
CTCAACTAGTCATTTTTTTCTCCTCTTCTTTTTTTTCATTATATCTAATTATTTTGCAGTTGGGCAACAGAGAAC
CATCCCTATTTTGTATTGAAGAGGGATTCACATCTGCATCTTAACTGCTCTTTATGAATGAAAAAACAGTCCTCT
GTATGTACTCCTCTTTACACTGGCCAGGGTCAGAGTTAAATAGAGTATATGCACTTTCCAAATTGGGGACAAGGG
CTCTAAAAAAAGCCCCAAAAGGAGAAGAACATCTGAGAACCTCCTCGGCCCTCCCAGTCCCTCGCTGCACAAATA
CTCCGCAAGAGAGGCCAGAATGACAGCTGACAGGGTCTATGGCCATCGGGTCGTCTCCGAAGATTTGGCAGGGGC
AGAAAACTCTGGCAGGCTTAAGATTTGGAATAAAGTCACAGAATTAAGGAAGCACCTCAATTTAGTTCAAACAAG
ACGCCAACATTCTCTCCACAGCTCACTTACCTCTCTGTGTTCAGATGTGGCCTTCCATTTATATGTGATCTTTGT
TTTATTAGTAAATGCTTATCATCTAAAGATGTAGCTCTGGCCCAGTGGGAAAAATTAGGAAGTGATTATAAATCG
AGAGGAGTTATAATAATCAAGATTAAATGTAAATAATCAGGGCAATCCCAACACATGTCTAGCTTTCACCTCCAG
GATCTATTGAGTGAACAGAATTGCAAATAGTCTCTATTTGTAATTGAACTTATCCTAAAACAAATAGTTTATAAA
TGTGAACTTAAACTCTAATTAATTCCAACTGTACTTTTAAGGCAGTGGCTGTTTTTAGACTTTCTTATCACTTAT
AGTTAGTAATGTACACCTACTCTATCAGAGAAAAACAGGAAAGGCTCGAAATACAAGCCATTCTAAGGAAATTAG
GGAGTCAGTTGAAATTCTATTCTGATCTTATTCTGTGGTGTCTTTTGCAGCCCAGACAAATGTGGTTACACACTT
TTTAAGAAATACAATTCTACATTGTCAAGCTTATGAAGGTTCCAATCAGATCTTTATTGTTATTCAATTTGGATC
TTTCAGGGATTTTTTTTTTAAATTATTATGGGACAAAGGACATTTGTTGGAGGGGTGGGAGGGAGGAAGAATTTT
TAAATGTAAAACATTCCCAAGTTTGGATCAGGGAGTTGGAAGTTTTCAGAATAACCAGAACTAAGGGTATGAAGG
ACCTGTATTGGGGTCGATGTGATGCCTCTGCGAAGAACCTTGTGTGACAAATGAGAAACATTTTGAAGTTTGTGG
TACGACCTTTAGATTCCAGAGACATCAGCATGGCTCAAAGTGCAGCTCCGTTTGGCAGTGCAATGGTATAAATTT
CAAGCTGGATATGTCTAATGGGTATTTAAACAATAAATGTGCAGTTTTAACTAACAGGATATTTAATGACAACCT
TCTGGTTGGTAGGGACATCTGTTTCTAAATGTTTATTATGTACAATACAGAAAAAAATTTTATAAAATTAAGCAA
TGTGAAACTGAATTGGAGAGTGATAATACAAGTCCTTTAGTCTTACCCAGTGAATCATTCTGTTCCATGTCTTTG
GACAACCATGACCTTGGACAATCATGAAATATGCATCTCACTGGATGCAAAGAAAATCAGATGGAGCATGAATGG
TACTGTACCGGTTCATCTGGACTGCCCCAGAAAAATAACTTCAAGCAAACATCCTATCAACAACAAGGTTGTTCT
GCATACCAAGCTGAGCACAGAAGATGGGAACACTGGTGGAGGATGGAAAGGCTCGCTCAATCAAGAAAATTCTGA
GACTATTAATAAATAAGACTGTAGTGTAGATACTGAGTAAATCCATGCACCTAAACCTTTTGGAAAATCTGCCGT
GGGCCCTCCAGATAGCTCATTTCATTAAGTTTTTCCCTCCAAGGTAGAATTTGCAAGAGTGACAGTGGATTGCAT
TTCTTTTGGGGAAGCTTTCTTTTGGTGGTTTTGTTTATTATACCTTCTTAAGTTTTCAACCAAGGTTTGCTTTTG
TTTTGAGTTACTGGGGTTATTTTTGTTTTAAATAAAAATAAGTGTACAATAAGTGTTTTTGTATTGAAAGCTTTT
GTTATCAAGATTTTCATACTTTTACCTTCCATGGCTCTTTTTAAGATTGATACTTTTAAGAGGTGGCTGATATTC
TGCAACACTGTACACATAAAAAATACGGTAAGGATACTTTACATGGTTAAGGTAAAGTAAGTCTCCAGTTGGCCA
CCATTAGCTATAATGGCACTTTGTTTGTGTTGTTGGAAAAAGTCACATTGCCATTAAACTTTCCTTGTCTGTCTA
GTTAATATTGTGAAGAAAAATAAAGTACAGTGTGAGATACTG.

“Bcl-X_{L}”: As used herein, the term “Bcl-X_{L}”, also known as Bcl-2-Like 1 and Bcl-2 Related Protein, Long Isoform, refers to a Bcl-X_{L} polypeptide or the gene encoding the polypeptide. A Bcl-X_{L} polypeptide is a multidomain, integral outer mitochondrial membrane protein that inhibits apoptosis. A nucleotide sequence encoding a human Bcl-X_{L} polypeptide is found in GenBank under Acc. No. NM_{—}138578.1. An exemplary human Bcl-X_{L} polypeptide sequence is found under Acc. No. NP_{—}612815.1. A genomic sequence which includes a human Bcl-X_{L} gene sequence is found under Acc. No. NC_{—}000020.9. “Bcl-X_{L}”, as used herein, includes human and non-human forms of Bcl-X_{L}. Sequences of non-human Bcl-X_{L} genes and polypeptides are known. For example, murine and rat Bcl-X_{L} polypeptide sequence are found under Acc. Nos. NP_{—}033873.3 and NP_{—}001028842.1, respectively. The GenBank database sequence entries above are incorporated herein by reference.

An amino acid sequence of a human Bcl-X_{L} polypeptide, found under GenBank Acc. No. NP_{—}612815.1, is as follows:

(SEQ ID NO: 3)
MSQSNRELVVDFLSYKLSQKGYSWSQFSDVEENRTEAPEGTESEMETPSAINGNPSWHLADSPAVNGATG
HSSSLDAREVIPMAAVKQALREAGDEFELRYRRAFSDLTSQLHITPGTAYQSFEQVVNELFRDGVNWGRI
VAFFSFGGALCVESVDKEMQVLVSRIAAWMATYLNDHLEPWIQENGGWDTFVELYGNNAAAESRKGQERF
NRWFLTGMTVAGVVLLGSLFSRK

A nucleotide sequence encoding a human Bcl-X_{L} polypeptide, found in GenBank under Acc. No. NM_{—}138578.1, is as follows:

(SEQ ID NO: 4)
GGAGGAGGAAGCAAGCGAGGGGGCTGGTTCCTGAGCTTCGCAATTCCTGTGTCGCCTTCTGGGCTCCCAG
CCTGCCGGGTCGCATGATCCCTCCGGCCGGAGCTGGTTTTTTTGCCAGCCACCGCGAGGCCGGCTGAGTT
ACCGGCATCCCCGCAGCCACCTCCTCTCCCGACCTGTGATACAAAAGATCTTCCGGGGGCTGCACCTGCC
TGCCTTTGCCTAAGGCGGATTTGAATCTCTTTCTCTCCCTTCAGAATCTTATCTTGGCTTTGGATCTTAG
AAGAGAATCACTAACCAGAGACGAGACTCAGTGAGTGAGCAGGTGTTTTGGACAATGGACTGGTTGAGCC
CATCCCTATTATAAAAATGTCTCAGAGCAACCGGGAGCTGGTGGTTGACTTTCTCTCCTACAAGCTTTCC
CAGAAAGGATACAGCTGGAGTCAGTTTAGTGATGTGGAAGAGAACAGGACTGAGGCCCCAGAAGGGACTG
AATCGGAGATGGAGACCCCCAGTGCCATCAATGGCAACCCATCCTGGCACCTGGCAGACAGCCCCGCGGT
GAATGGAGCCACTGGCCACAGCAGCAGTTTGGATGCCCGGGAGGTGATCCCCATGGCAGCAGTAAAGCAA
GCGCTGAGGGAGGCAGGCGACGAGTTTGAACTGCGGTACCGGCGGGCATTCAGTGACCTGACATCCCAGC
TCCACATCACCCCAGGGACAGCATATCAGAGCTTTGAACAGGTAGTGAATGAACTCTTCCGGGATGGGGT
AAACTGGGGTCGCATTGTGGCCTTTTTCTCCTTCGGCGGGGCACTGTGCGTGGAAAGCGTAGACAAGGAG
ATGCAGGTATTGGTGAGTCGGATCGCAGCTTGGATGGCCACTTACCTGAATGACCACCTAGAGCCTTGGA
TCCAGGAGAACGGCGGCTGGGATACTTTTGTGGAACTCTATGGGAACAATGCAGCAGCCGAGAGCCGAAA
GGGCCAGGAACGCTTCAACCGCTGGTTCCTGACGGGCATGACTGTGGCCGGCGTGGTTCTGCTGGGCTCA
CTCTTCAGTCGGAAATGACCAGACACTGACCATCCACTCTACCCTCCCACCCCCTTCTCTGCTCCACCAC
ATCCTCCGTCCAGCCGCCATTGCCACCAGGAGAACCACTACATGCAGCCCATGCCCACCTGCCCATCACA
GGGTTGGGCCCAGATCTGGTCCCTTGCAGCTAGTTTTCTAGAATTTATCACACTTCTGTGAGACCCCCAC
ACCTCAGTTCCCTTGGCCTCAGAATTCACAAAATTTCCACAAAATCTGTCCAAAGGAGGCTGGCAGGTAT
GGAAGGGTTTGTGGCTGGGGGCAGGAGGGCCCTACCTGATTGGTGCAACCCTTACCCCTTAGCCTCCCTG
AAAATGTTTTTCTGCCAGGGAGCTTGAAAGTTTTCAGAACCTCTTCCCCAGAAAGGAGACTAGATTGCCT
TTGTTTTGATGTTTGTGGCCTCAGAATTGATCATTTTCCCCCCACTCTCCCCACACTAACCTGGGTTCCC
TTTCCTTCCATCCCTACCCCCTAAGAGCCATTTAGGGGCCACTTTTGACTAGGGATTCAGGCTGCTTGGG
ATAAAGATGCAAGGACCAGGACTCCCTCCTCACCTCTGGACTGGCTAGAGTCCTCACTCCCAGTCCAAAT
GTCCTCCAGAAGCCTCTGGCTAGAGGCCAGCCCCACCCAGGAGGGAGGGGGCTATAGCTACAGGAAGCAC
CCCATGCCAAAGCTAGGGTGGCCCTTGCAGTTCAGCACCACCCTAGTCCCTTCCCCTCCCTGGCTCCCAT
GACCATACTGAGGGACCAACTGGGCCCAAGACAGATGCCCCAGAGCTGTTTATGGCCTCAGCTGCCTCAC
TTCCTACAAGAGCAGCCTGTGGCATCTTTGCCTTGGGCTGCTCCTCATGGTGGGTTCAGGGGACTCAGCC
CTGAGGTGAAAGGGAGCTATCAGGAACAGCTATGGGAGCCCCAGGGTCTTCCCTACCTCAGGCAGGAAGG
GCAGGAAGGAGAGCCTGCTGCATGGGGTGGGGTAGGGCTGACTAGAAGGGCCAGTCCTGCCTGGCCAGGC
AGATCTGTGCCCCATGCCTGTCCAGCCTGGGCAGCCAGGCTGCCAAGGCCAGAGTGGCCTGGCCAGGAGC
TCTTCAGGCCTCCCTCTCTCTTCTGCTCCACCCTTGGCCTGTCTCATCCCCAGGGGTCCCAGCCACCCCG
GGCTCTCTGCTGTACATATTTGAGACTAGTTTTTATTCCTTGTGAAGATGATATACTATTTTTGTTAAGC
GTGTCTGTATTTATGTGTGAGGAGCTGCTGGCTTGCAGTGCGCGTGCACGTGGAGAGCTGGTGCCCGGAG
ATTGGACGGCCTGATGCTCCCTCCCCTGCCCTGGTCCAGGGAAGCTGGCCGAGGGTCCTGGCTCCTGAGG
GGCATCTGCCCCTCCCCCAACCCCCACCCCACACTTGTTCCAGCTCTTTGAAATAGTCTGTGTGAAGGTG
AAAGTGCAGTTCAGTAATAAACTGTGTTTACTCAGTGAAAAAAAAAAAAAAAAAA

“Depsipeptide”: The term “depsipeptide”, as used herein, refers to polypeptides that contain both ester and amide bonds. Naturally occurring depsipeptides are usually cyclic. Some depsipeptides have been shown to have potent antibiotic activity. Examples of depsipeptides include actinomycin, enniatins, valinomycin, and romidepsin.

“Effective amount”: In general, the “effective amount” of an active agent or combination of agents refers to an amount sufficient to elicit the desired biological response. As will be appreciated by those of ordinary skill in this art, the effective amount of an agent may vary depending on such factors as the desired biological endpoint, the pharmacokinetics of the agent being delivered, the disease being treated, the mode of administration, and the patient. For example, the effective amount of an agent (e.g., romidepsin) is the amount that results in reducing the tumor burden, causing a remission, or curing a patient.

“Expression”: The terms “express” and “expression”, as used herein to refer to gene expression, include expression of nucleic acids (e.g., mRNA) and expression of polypeptides. Thus, “Bcl-2 expression” can be determined by evaluating expression of Bcl-2 mRNA and/or expression of Bcl-2 polypeptides.

“Overexpression”: As used herein, a cancer cell which “overexpresses” a gene indicates that expression of the gene is significantly higher as compared to a noncancerous cell, e.g., a noncancerous cell of the same tissue type. A population of cancer cells “overexpresses” a gene if expression of the gene is significantly higher, and/or if the percentage of cells that expresses the gene is significantly higher (e.g., at least 10%, 25%, 50%, 100%, 200%, 300%, 400%, or 500% higher), as compared to noncancerous cells (e.g., noncancerous cells of the same tissue type). Overexpression can be determined by comparing expression in a cancer cell to a reference. In some embodiments, the reference is expression of the gene in a noncancerous cell (e.g., a noncancerous cell of the same tissue type). In some embodiments, the reference is expression of a different gene in the cancer cell. In some embodiments, the reference is expression of the gene in a cell line (e.g., a cell line which is known to lack expression, or which overexpresses the gene). Overexpression may be caused by gene amplification or by increased transcription or translation of the gene. Overexpression can be determined in an assay that evaluates polypeptides within a cell, secreted by a cell, or expressed on the cell surface (where applicable) (e.g., by immunohistochemistry, Western blotting, or FACS, e.g., intracellular FACS staining) or in an assay that evaluates nucleic acids such as mRNA (e.g., in situ hybridization, microarray analysis, Southern blotting, Northern blotting, or PCR-based methods, such as QTPCR).

“P-glycoprotein”: As used herein, “P-glycoprotein” (also known as P-gp, Gp170, ATP-Binding Cassette, Subfamily B, Member 1, and ABCB1) is an ATP-binding cassette transporter, which is a large transmembrane protein. Human P-glycoprotein is encoded by the MDR1 gene. Expression of P-glycoprotein can be determined by evaluating expression of MDR1 nucleic acids, or by evaluating expression of P-glycoprotein polypeptides. An amino acid sequence of a human P-glycoprotein polypeptide, found under GenBank Acc. No. NP_{—}000918.2, is as follows:

(SEQ ID NO: 5)
MDLEGDRNGGAKKKNFFKLNNKSEKDKKEKKPTVSVFSMFRYSNWLDKLYMVVGTLAAIIHGAGLPLMML
VFGEMTDIFANAGNLEDLMSNITNRSDINDTGFFMNLEEDMTRYAYYYSGIGAGVLVAAYIQVSFWCLAA
GRQIHKIRKQFFHAIMRQEIGWFDVHDVGELNTRLTDDVSKINEGIGDKIGMFFQSMATFFTGFIVGFTR
GWKLTLVILAISPVLGLSAAVWAKILSSFTDKELLAYAKAGAVAEEVLAAIRTVIAFGGQKKELERYNKN
LEEAKRIGIKKAITANISIGAAFLLIYASYALAFWYGTTLVLSGEYSIGQVLTVFFSVLIGAFSVGQASP
SIEAFANARGAAYEIFKIIDNKPSIDSYSKSGHKPDNIKGNLEFRNVHFSYPSRKEVKILKGLNLKVQSG
QTVALVGNSGCGKSTTVQLMQRLYDPTEGMVSVDGQDIRTINVRFLREIIGVVSQEPVLFATTIAENIRY
GRENVTMDEIEKAVKEANAYDFIMKLPHKFDTLVGERGAQLSGGQKQRIAIARALVRNPKILLLDEATSA
LDTESEAVVQVALDKARKGRTTIVIAHRLSTVRNADVIAGFDDGVIVEKGNHDELMKEKGIYFKLVTMQT
AGNEVELENAADESKSEIDALEMSSNDSRSSLIRKRSTRRSVRGSQAQDRKLSTKEALDESIPPVSFWRI
MKLNLTEWPYFVVGVFCAIINGGLQPAFAIIFSKIIGVFTRIDDPETKRQNSNLFSLLFLALGIISFITF
FLQGFTFGKAGEILTKRLRYMVERSMLRQDVSWFDDPKNTTGALTTRLANDAAQVKGAIGSRLAVITQNI
ANLGTGIIISFIYGWQLTLLLLAIVPIIAIAGVVEMKMLSGQALKDKKELEGSGKIATEAIENFRTVVSL
TQEQKFEHMYAQSLQVPYRNSLRKAHIFGITFSFTQAMMYFSYAGCFRFGAYLVAHKLMSFEDVLLVFSA
VVFGAMAVGQVSSFAPDYAKAKISAAHIIMIIEKTPLIDSYSTEGLMPNTLEGNVTFGEVVFNYPTRPDI
PVLQGLSLEVKKGQTLALVGSSGCGKSTVVQLLERFYDPLAGKVLLDGKEIKRLNVQWLRAHLGIVSQEP
ILFDCSIAENIAYGDNSRVVSQEEIVRAAKEANIHAFIESLPNKYSTKVGDKGTQLSGGQKQRIAIARAL
VRQPHILLLDEATSALDTESEKVVQEALDKAREGRTCIVIAHRLSTIQNADLIVVFQNGRVKEHGTHQQL
LAQKGIYFSMVSVQAGTKRQ

A nucleotide sequence encoding a human P-glycoprotein polypeptide, found in GenBank under Acc. No. NM_{—}000927.3, is as follows:

(SEQ ID NO: 6)
TATTCAGATATTCTCCAGATTCCTAAAGATTAGAGATCATTTCTCATTCTCCTAGGAGTACTCACTTCAG
GAAGCAACCAGATAAAAGAGAGGTGCAACGGAAGCCAGAACATTCCTCCTGGAAATTCAACCTGTTTCGC
AGTTTCTCGAGGAATCAGCATTCAGTCAATCCGGGCCGGGAGCAGTCATCTGTGGTGAGGCTGATTGGCT
GGGCAGGAACAGCGCCGGGGCGTGGGCTGAGCACAGCCGCTTCGCTCTCTTTGCCACAGGAAGCCTGAGC
TCATTCGAGTAGCGGCTCTTCCAAGCTCAAAGAAGCAGAGGCCGCTGTTCGTTTCCTTTAGGTCTTTCCA
CTAAAGTCGGAGTATCTTCTTCCAAAATTTCACGTCTTGGTGGCCGTTCCAAGGAGCGCGAGGTCGGAAT
GGATCTTGAAGGGGACCGCAATGGAGGAGCAAAGAAGAAGAACTTTTTTAAACTGAACAATAAAAGTGAA
AAAGATAAGAAGGAAAAGAAACCAACTGTCAGTGTATTTTCAATGTTTCGCTATTCAAATTGGCTTGACA
AGTTGTATATGGTGGTGGGAACTTTGGCTGCCATCATCCATGGGGCTGGACTTCCTCTCATGATGCTGGT
GTTTGGAGAAATGACAGATATCTTTGCAAATGCAGGAAATTTAGAAGATCTGATGTCAAACATCACTAAT
AGAAGTGATATCAATGATACAGGGTTCTTCATGAATCTGGAGGAAGACATGACCAGGTATGCCTATTATT
ACAGTGGAATTGGTGCTGGGGTGCTGGTTGCTGCTTACATTCAGGTTTCATTTTGGTGCCTGGCAGCTGG
AAGACAAATACACAAAATTAGAAAACAGTTTTTTCATGCTATAATGCGACAGGAGATAGGCTGGTTTGAT
GTGCACGATGTTGGGGAGCTTAACACCCGACTTACAGATGATGTCTCCAAGATTAATGAAGGAATTGGTG
ACAAAATTGGAATGTTCTTTCAGTCAATGGCAACATTTTTCACTGGGTTTATAGTAGGATTTACACGTGG
TTGGAAGCTAACCCTTGTGATTTTGGCCATCAGTCCTGTTCTTGGACTGTCAGCTGCTGTCTGGGCAAAG
ATACTATCTTCATTTACTGATAAAGAACTCTTAGCGTATGCAAAAGCTGGAGCAGTAGCTGAAGAGGTCT
TGGCAGCAATTAGAACTGTGATTGCATTTGGAGGACAAAAGAAAGAACTTGAAAGGTACAACAAAAATTT
AGAAGAAGCTAAAAGAATTGGGATAAAGAAAGCTATTACAGCCAATATTTCTATAGGTGCTGCTTTCCTG
CTGATCTATGCATCTTATGCTCTGGCCTTCTGGTATGGGACCACCTTGGTCCTCTCAGGGGAATATTCTA
TTGGACAAGTACTCACTGTATTCTTTTCTGTATTAATTGGGGCTTTTAGTGTTGGACAGGCATCTCCAAG
CATTGAAGCATTTGCAAATGCAAGAGGAGCAGCTTATGAAATCTTCAAGATAATTGATAATAAGCCAAGT
ATTGACAGCTATTCGAAGAGTGGGCACAAACCAGATAATATTAAGGGAAATTTGGAATTCAGAAATGTTC
ACTTCAGTTACCCATCTCGAAAAGAAGTTAAGATCTTGAAGGGTCTGAACCTGAAGGTGCAGAGTGGGCA
GACGGTGGCCCTGGTTGGAAACAGTGGCTGTGGGAAGAGCACAACAGTCCAGCTGATGCAGAGGCTCTAT
GACCCCACAGAGGGGATGGTCAGTGTTGATGGACAGGATATTAGGACCATAAATGTAAGGTTTCTACGGG
AAATCATTGGTGTGGTGAGTCAGGAACCTGTATTGTTTGCCACCACGATAGCTGAAAACATTCGCTATGG
CCGTGAAAATGTCACCATGGATGAGATTGAGAAAGCTGTCAAGGAAGCCAATGCCTATGACTTTATCATG
AAACTGCCTCATAAATTTGACACCCTGGTTGGAGAGAGAGGGGCCCAGTTGAGTGGTGGGCAGAAGCAGA
GGATCGCCATTGCACGTGCCCTGGTTCGCAACCCCAAGATCCTCCTGCTGGATGAGGCCACGTCAGCCTT
GGACACAGAAAGCGAAGCAGTGGTTCAGGTGGCTCTGGATAAGGCCAGAAAAGGTCGGACCACCATTGTG
ATAGCTCATCGTTTGTCTACAGTTCGTAATGCTGACGTCATCGCTGGTTTCGATGATGGAGTCATTGTGG
AGAAAGGAAATCATGATGAACTCATGAAAGAGAAAGGCATTTACTTCAAACTTGTCACAATGCAGACAGC
AGGAAATGAAGTTGAATTAGAAAATGCAGCTGATGAATCCAAAAGTGAAATTGATGCCTTGGAAATGTCT
TCAAATGATTCAAGATCCAGTCTAATAAGAAAAAGATCAACTCGTAGGAGTGTCCGTGGATCACAAGCCC
AAGACAGAAAGCTTAGTACCAAAGAGGCTCTGGATGAAAGTATACCTCCAGTTTCCTTTTGGAGGATTAT
GAAGCTAAATTTAACTGAATGGCCTTATTTTGTTGTTGGTGTATTTTGTGCCATTATAAATGGAGGCCTG
CAACCAGCATTTGCAATAATATTTTCAAAGATTATAGGGGTTTTTACAAGAATTGATGATCCTGAAACAA
AACGACAGAATAGTAACTTGTTTTCACTATTGTTTCTAGCCCTTGGAATTATTTCTTTTATTACATTTTT
CCTTCAGGGTTTCACATTTGGCAAAGCTGGAGAGATCCTCACCAAGCGGCTCCGATACATGGTTTTCCGA
TCCATGCTCAGACAGGATGTGAGTTGGTTTGATGACCCTAAAAACACCACTGGAGCATTGACTACCAGGC
TCGCCAATGATGCTGCTCAAGTTAAAGGGGCTATAGGTTCCAGGCTTGCTGTAATTACCCAGAATATAGC
AAATCTTGGGACAGGAATAATTATATCCTTCATCTATGGTTGGCAACTAACACTGTTACTCTTAGCAATT
GTACCCATCATTGCAATAGCAGGAGTTGTTGAAATGAAAATGTTGTCTGGACAAGCACTGAAAGATAAGA
AAGAACTAGAAGGTTCTGGGAAGATCGCTACTGAAGCAATAGAAAACTTCCGAACCGTTGTTTCTTTGAC
TCAGGAGCAGAAGTTTGAACATATGTATGCTCAGAGTTTGCAGGTACCATACAGAAACTCTTTGAGGAAA
GCACACATCTTTGGAATTACATTTTCCTTCACCCAGGCAATGATGTATTTTTCCTATGCTGGATGTTTCC
GGTTTGGAGCCTACTTGGTGGCACATAAACTCATGAGCTTTGAGGATGTTCTGTTAGTATTTTCAGCTGT
TGTCTTTGGTGCCATGGCCGTGGGGCAAGTCAGTTCATTTGCTCCTGACTATGCCAAAGCCAAAATATCA
GCAGCCCACATCATCATGATCATTGAAAAAACCCCTTTGATTGACAGCTACAGCACGGAAGGCCTAATGC
CGAACACATTGGAAGGAAATGTCACATTTGGTGAAGTTGTATTCAACTATCCCACCCGACCGGACATCCC
AGTGCTTCAGGGACTGAGCCTGGAGGTGAAGAAGGGCCAGACGCTGGCTCTGGTGGGCAGCAGTGGCTGT
GGGAAGAGCACAGTGGTCCAGCTCCTGGAGCGGTTCTACGACCCCTTGGCAGGGAAAGTGCTGCTTGATG
GCAAAGAAATAAAGCGACTGAATGTTCAGTGGCTCCGAGCACACCTGGGCATCGTGTCCCAGGAGCCCAT
CCTGTTTGACTGCAGCATTGCTGAGAACATTGCCTATGGAGACAACAGCCGGGTGGTGTCACAGGAAGAG
ATTGTGAGGGCAGCAAAGGAGGCCAACATACATGCCTTCATCGAGTCACTGCCTAATAAATATAGCACTA
AAGTAGGAGACAAAGGAACTCAGCTCTCTGGTGGCCAGAAACAACGCATTGCCATAGCTCGTGCCCTTGT
TAGACAGCCTCATATTTTGCTTTTGGATGAAGCCACGTCAGCTCTGGATACAGAAAGTGAAAAGGTTGTC
CAAGAAGCCCTGGACAAAGCCAGAGAAGGCCGCACCTGCATTGTGATTGCTCACCGCCTGTCCACCATCC
AGAATGCAGACTTAATAGTGGTGTTTCAGAATGGCAGAGTCAAGGAGCATGGCACGCATCAGCAGCTGCT
GGCACAGAAAGGCATCTATTTTTCAATGGTCAGTGTCCAGGCTGGAACAAAGCGCCAGTGAACTCTGACT
GTATGAGATGTTAAATACTTTTTAATATTTGTTTAGATATGACATTTATTCAAAGTTAAAAGCAAACACT
TACAGAATTATGAAGAGGTATCTGTTTAACATTTCCTCAGTCAAGTTCAGAGTCTTCAGAGACTTCGTAA
TTAAAGGAACAGAGTGAGAGACATCATCAAGTGGAGAGAAATCATAGTTTAAACTGCATTATAAATTTTA
TAACAGAATTAAAGTAGATTTTAAAAGATAAAATGTGTAATTTTGTTTATATTTTCCCATTTGGACTGTA
ACTGACTGCCTTGCTAAAAGATTATAGAAGTAGCAAAAAGTATTGAAATGTTTGCATAAAGTGTCTATAA
TAAAACTAAACTTTCATGTGACTGGAGTCATCTTGTCCAAACTGCCTGTGAATATATCTTCTCTCAATTG
GAATATTGTAGATAACTTCTGCTTTAAAAAAGTTTTCTTTAAATATACCTACTCATTTTTGTGGGAATGG
TTAAGCAGTTTAAATAATTCCTGTTGTATATGTCTATTCACATTGGGTCTTACAGAACCATCTGGCTTCA
TTCTTCTTGGACTTGATCCTGCTGATTCTTGCATTTCCACAT

“Peptide” or “protein” or “polypeptide”: According to the present invention, a “peptide” or “protein” or “polypeptide” comprises a string of at least three amino acids linked together by peptide bonds. The terms “protein”, “peptide”, and “polypeptide” may be used interchangeably. Peptides preferably contain only natural amino acids, although non-natural amino acids (L e., compounds that do not occur in nature but that can be incorporated into a polypeptide chain) and/or amino acid analogs as are known in the art may alternatively be employed. Also, one or more of the amino acids in a peptide may be modified, for example, by the addition of a chemical entity such as a carbohydrate group, a phosphate group, a farnesyl group, an isofarnesyl group, a fatty acid group, a linker for conjugation, functionalization, or other modification, etc. In certain embodiments, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide. In certain embodiments, peptide refers to depsipeptide.

“Romidepsin”: The term “romidepsin”, refers to a natural product of the chemical structure:

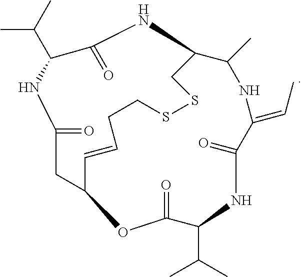

Romidepsin is a deacetylase inhibitor and is also known in the art by the names FK228, FR901228, NSC630176, or depsipeptide. The identification and preparation of romidepsin is described in U.S. Pat. No. 4,977,138, issued Dec. 11, 1990, which is incorporated herein by reference. The molecular formula is C₂₄H₃₆N₄O₆S₂; and the molecular weight is 540.71 g/mol. Romidepsin has the chemical name, (1S,4S,10S,16E,21R)-7-[(2Z)-ethylidene]-4,21-diisopropyl-2-oxa-12,13-dithia-5,8,20,23-tetraazabicyclo[8.7.6]tricos-16-ene-3,6,9,19,22-pentanone. Romidepsin has been assigned the CAS number 128517-07-7. In crystalline form, romidepsin is typically a white to pale yellowish white crystal or crystalline powder. The term “romidepsin” encompasses this compound and any pharmaceutically forms thereof In certain embodiments, the term “romidepsin” may also include salts, pro-drugs, esters, protected forms, reduced forms, oxidized forms, isomers, stereoisomers (e.g., enantiomers, diastereomers), tautomers, and derivatives thereof.

“Sample”: A sample refers to a sample obtained from a subject. The sample may be from any biological tissue or fluid. In some embodiments, a sample is derived from a human, e.g., a patient, e.g., a cancer patient. Samples include tissues, sections of tissues, cells, fluids, or extracts thereof, and can be isolated by any means (e.g., from blood, serum, biopsy, lymph node biopsy, bone marrow biopsy, needle biopsy, aspiration, etc.).

“Treating”: “Treating” or “treatment” refers to both therapeutic treatment and prophylactic or preventative measures, wherein the object is to prevent, slow down (lessen), or alleviate cancer or a cancer symptom. In some embodiments, a subject is successfully “treated” for a cancer if, after receiving a therapeutically effective amount of an agent (e.g., romidepsin), the subject shows an observable and/or measurable reduction in or absence of one or more of the following: reduction in the number of cancer cells (e.g., by apoptosis) or absence of the cancer cells; reduction in the tumor size; inhibition of cancer cell infiltration into peripheral organs or tissues; inhibition of tumor metastasis; inhibition, to some extent, of tumor growth; and/or relief to some extent, one or more of the symptoms associated with the specific cancer; and reduced morbidity and mortality.

“Underexpresses”: As used herein, a cancer cell which “underexpresses” a gene indicates that expression of the gene is significantly lower as compared to a noncancerous cell, e.g., a noncancerous cell of the same tissue type. A population of cancer cells “underexpresses” a gene if expression of the gene is significantly lower, and/or if the percentage of cells that expresses the gene is significantly lower (e.g., two-fold, three-fold, four-fold, or five-fold less), as compared to noncancerous cells (e.g., noncancerous cells of the same tissue type). Underexpression can be determined by comparing expression in a cancer cell to a reference. In some embodiments, the reference is expression of the gene in a noncancerous cell (e.g., a noncancerous cell of the same tissue type). In some embodiments, the reference is expression of a different gene in the cancer cell. In some embodiments, the reference is expression of the gene in a cell line (e.g., a cell line which is known to lack expression, or which overexpresses the gene). Underexpression can be determined in an assay that evaluates polypeptides within a cell, secreted by a cell, or expressed on the cell surface (where applicable) (e.g., by immunohistochemistry or FACS) or in an assay that evaluates nucleic acids such as mRNA (e.g., in situ hybridization, Southern blotting, Northern blotting, or PCR-based methods).

### BRIEF DESCRIPTION OF THE DRAWINGS

FIG. 1. Eμ-myc lymphomas overexpressing Bcl-2 are resistant to romidepsin in vitro in short-term assays. 4242Eμ-myc, 4242Eμ-myc/Bcl-2, 229Eμmyc, 229Eμ-myc/Bcl-2, 226Eμ-myc, and 226Eμ-myc/Bcl-2 lymphomas were incubated with the indicated concentrations of romidepsin or oxamflatin for 24 h. Cell viability was assessed by (FIG. 1A) propidium iodide staining and (FIG. 1B) loss of MOMP. Bars, SE of at least three independent experiments.

FIG. 2. Romidepsin can kill Eμ-myc/Bcl-2 lymphomas over time. 4242Eμ-myc, 4242Eμ-myc/Bcl-2, 229Eμ-myc, 229Eμ-myc/Bcl-2, 226Eμ-myc, 226Eμmyc/Bcl-2, 102Eμ-myc, and 102Eμ-myc/Bcl-2 lymphomas were incubated for up to 72 h with the concentration of HDACi required to kill ˜70% of Eμ-myc lymphomas following 24-h treatment (3 nmol/L romidepsin or 0.1 μmol/L oxamflatin). Cell viability was assessed by (FIG. 2A) propidium iodide staining and (FIG. 2B) loss of MOMP. Bars, SE of at least three independent experiments. FIG. 2C, 4242Eμ-myc cells were treated with 3.0 nmol/L romidepsin, 0.1 μmol/L oxamflatin or vehicle (lanes 7-9) for 2 h (lanes 1, 4, and 7), 8 h (lanes 2, 5, and 8), and 24 h (lanes 3, 6, and 9). Whole-cell lysates were used for Western blot analysis using antibodies specific for acetylated histones H3 and H4. Blots were reprobed with anti-tubulin polyclonal antibody to assess protein loading. FIG. 2D, 4242Eμ-myc/Bcl-2 and 226Eμ-myc/Bcl-2 cells were treated with 3.0 nmol/L romidepsin for 2 h (lanes 1 and 4), 8 h (lanes 2 and 5), and 24 h or vehicle for 24 h (lanes 7 and 8). Whole-cell lysates were used for Western blot analysis using antibodies specific for acetylated histones H3 and H4. Blots were reprobed with anti-β actin polyclonal antibody to assess protein loading.

FIG. 3. Romidepsin can kill Eμ-myc/Bcl-2 lymphomas in vivo. C57BL/6 mice bearing (FIG. 3A) 4242Eμ-myc, (FIG. 3B) 229Eμ-myc, (FIG. 3C) 226Eμ-myc, (FIG. 3D) 102Eμ-myc, (FIG. 3E) 4242Eμ-myc/Bcl-2, (FIG. 3F) 229Eμ-myc/Bcl-2, (FIG. 3G) 226Eμ-myc/Bcl-2, and (FIG. 3H) 102Eμ-myc/Bcl-2 lymphomas were injected with romidepsin (5.6 mg/kg i.v.) or vehicle. Lymphoma cells were harvested at the time points (hours) indicated following romidepsin treatment or 24 h following vehicle treatment (v). Apoptosis was measured by either Fluorogold staining for outer cell membrane permeabilization (gray columns) or DNA fragmentation (white columns).

FIG. 4. Therapeutic effect of romidepsin in vivo. C57BL/6 mice (10 mice per group) bearing (FIG. 4A) 4242Eμ-myc, (FIG. 4B) 229Eμ-myc, (FIG. 4C) 226Eμ-myc, (FIG. 4D) 102Eμ-myc, (FIG. 4E) 4242Eμ-myc/Bcl-2, (FIG. 4F) 229Eμmyc/Bcl-2, (FIG. 4G) 226Eμ-myc/Bcl-2, and (FIG. 4H) 102Eμ-myc/Bcl-2, lymphomas were treated with romidepsin or vehicle. Therapy commenced after WBC counts reached ≧13×10³/μL. Therapy consisted of either 5.6 mg/kg romidepsin (injected i.v. every 4 d for a total of four doses) or vehicle. Kaplan-Meier survival curves of vehicle-treated mice (dashed line) and romidepsin-treated mice (solid line) are shown. Median survival and P values for the different lymphomas were as follows: 4242Eμ-myc, median survival vehicle 19 d, median survival romidepsin 28 d, P<0.0003; 4242Eμ-myc/Bcl-2, median survival vehicle 12 d, median survival romidepsin 22.5 d, P<0.0001; 229Eμ-myc, median survival vehicle 20 days, median survival romidepsin 30 d, P<0.0001; 229Eμ-myc/Bcl-2, median survival vehicle 18 d, median survival romidepsin 30 d, P<0.0001; 226Eμ-myc, median survival vehicle 15 d, median survival romidepsin 19.5 d, P<0.0001; 226Eμ-myc/Bcl-2, median survival vehicle 16 d, median survival romidepsin 16 d, P=0.86; 102Eμmyc, median survival vehicle 14 d, median survival romidepsin 22 d, P<0.0001; 102Eμ-myc/Bcl-2, median survival vehicle 11 d, median survival romidepsin 14.5 d, P<0.07.

FIG. 5. Expression of exogenous Bcl-2 and endogenous prosurvival Bcl-2 family proteins in Eμ-myc and Eμ-myc/Bcl-2 lymphomas. FIG. 5A, expression of exogenous Bcl-2 was detected by Western blot using whole-cell lysates from 4242Eμ-myc, 4242Eμ-myc/Bcl-2, 2294Eμ-myc, 229Eμ-myc/Bcl-2, 226Eμ-myc, 226Eμ-myc/Bcl-2, 102Eμ-myc, and 102Eμ-myc/Bcl-2 lymphomas. Blots were reprobed with anti-tubulin polyclonal antibody to assess protein loading. FIG. 5B, expression of endogenous Bcl-X_{L}, Mcl-1, Bcl-w, and A1 was detected by Western blot using whole-cell lysates from 4242Eμ-myc/Bcl-2, 229Eμ-myc/Bcl-2, 226Eμ-myc/Bcl-2, and 102Eμ-myc/Bcl-2 lymphomas. Blots were reprobed with anti-tubulin polyclonal antibody to assess protein loading.

FIG. 6. Eμ-myc lymphomas overexpressing Bcl-X_{L} are resistant to romidepsin and oxamflatin in vitro. 4242Eμ-myc and 4242Eμ-myc/Bcl-X_{L} were incubated with the indicated concentrations of (FIG. 6A) romidepsin or (FIG. 6B) oxamflatin for 24 h or with (FIG. 6C) 3 nmol/L romidepsin or (FIG. 6D) 0.1 μmol/L oxamflatin for up to 72 h. Cell viability was assessed by propidium iodide staining and by loss of MOMP. Bars, SE of at least three independent experiments.

### DETAILED DESCRIPTION OF CERTAIN EMBODIMENTS OF THE INVENTION

The present invention provides novel methods for treating cancers, such as lymphomas, based on expression of anti-apoptotic factors. More particularly, the invention provides methods of treating cancers identified as expressing Bcl-2 and/or which do not overexpress Bcl-X_{L}, with romidepsin. Use of romidepsin for treating Bcl-2⁺ cancers, and for treating cancers that do not overexpress Bcl-X_{L}, arises from the discovery that romidepsin is effective in inducing apoptosis of Bcl-2-overexpressing cells in vitro and in vivo (see Examples 1-4 herein). Treatment of Bcl-2⁺ tumors with romidepsin was shown to provide a therapeutic benefit in vivo. (see Example 2 herein). Romidepsin treatment of Bcl-2⁺ tumors is particularly effective when the tumor does not overexpress Bcl-X_{L}, and when the tumor does not overexpress P-glycoprotein. The finding that Bcl-2 does not suppress apoptotic and therapeutic activities of romidepsin reveals romidepsin a uniquely effective agent for treating cancers that express, or overexpress, Bcl-2.

Gene Expression and Selection of Subjects for Treatment with Romidepsin

Bcl-2 prolongs cell survival by inhibiting apoptosis. Dysregulation of Bcl-2 expression is thought to contribute to the development, persistence, and drug resistance of certain cancers. The treatment methods herein are based, in part, on the surprising discovery that Bcl-2 does not suppress apoptotic and therapeutic effects of romidepsin. Romidepsin is effective for treating cancers that are positive for expression of Bcl-2, including cancers that overexpress Bcl-2. It has also been discovered that romidepsin therapy is effective for treating tumors that do not overexpress Bcl-X_{L} or P-glycoprotein.

According to methods described herein, treatment with romidepsin is indicated for a subject having a cancer (e.g., a lymphoma) that expresses (e.g., overexpresses) Bcl-2. The subject may be identified as having a Bcl-2⁺ cancer by any available means. In some embodiments, a subject is selected for treatment with romidepsin, wherein the subject has already been identified as having a Bcl-2⁺ cancer. In some embodiments, a method of treatment includes analysis of Bcl-2 expression in cells of the cancer (e.g., prior to treatment with romidepsin, during a course of treatment with romidepsin, and/or after treatment with romidepsin). In some embodiments, cells of the cancer have a chromosomal rearrangement that produces a translocation of a Bcl-2 gene (e.g., a human t(14;18) chromosomal translocation that places the Bcl-2 gene under the transcriptional control of the immunoglobulin heavy chain locus).

In some embodiments, Bcl-2 expression is determined by analyzing Bcl-2 mRNA expression (e.g., using PCR, e.g., reverse transcription-PCR (RT-PCR), Northern blot analysis, microarray analysis, or in situ hybridization). In some embodiments, Bcl-2 expression is determined by analyzing Bcl-2 polypeptide expression (e.g., using an antibody-based technique, such as immunohistochemistry, Western blot, or FACS analysis). Bcl-2 expression can also be determined indirectly, e.g., by detecting the presence of a chromosomal translocation that results in Bcl-2 expression or overexpression (see, e.g., Gribben et al. (Blood 78(12):3275-3280, 1991), which describes a PCR-based method for detecting Bcl-2 gene rearrangements).

In some embodiments, Bcl-2 expression is determined and compared to a reference (e.g., a reference sample, or a reference value, comparison to which indicates whether or not the cancer expresses or overexpresses Bcl-2). In some embodiments, Bcl-2 expression in cells of a cancer is determined, relative to Bcl-2 expression in cells of a non-cancerous tissue, e.g., a non-cancerous tissue of the same tissue type as the tumor. In some embodiments, Bcl-2 expression in a lymphoma is determined, relative to Bcl-2 expression in non-cancerous lymphocytes. In some embodiments, the percentage of Bcl-2⁺ cells in a sample from a cancer are determined Methods of analyzing and quantitating Bcl-2 expression in patient samples, primary cells, and cell lines by immunofluorescence, immunohistochemistry, and other methods, are described, e.g., in Campos et al., Blood 81(11):3091-3096, 1993; Pezzella et al., Am. J. Pathol. 137(2):225-32, 1990; Swerdlow et al., Leukemia 7:1456-1458, 1993; and Porwit-Macdonald et al., Leukemia 9(7):1191-8, 1995.

In some embodiments, methods of treating a subject with romidepsin include methods in which the subject has a cancer that does not overexpress Bcl-X_{L} (e.g., the cancer expresses Bcl-X_{L} at low levels, or the cancer lacks expression of Bcl-X_{L}). The subject may be identified as one whose cancer lacks overexpression of Bcl-X_{L} by any available means. In some embodiments, a subject is selected for treatment with romidepsin, wherein the subject has already been identified as having a cancer that does not overexpress Bcl-X_{L}. In some embodiments, a method of treatment includes analysis of Bcl-X_{L} expression in cells of the cancer (e.g., prior to treatment with romidepsin, during a course of treatment with romidepsin, and/or after treatment with romidepsin). In some embodiments, the cancer is a cancer that overexpresses Bcl-2.

Bcl-X_{L} expression can be determined by means such as those mentioned above with respect to Bcl-2, e.g., by analyzing Bcl-X_{L} mRNA expression (e.g., PCR, Northern blot analysis, or in situ hybridization) or Bcl-X_{L} polypeptide expression (e.g., using immunohistochemistry, Western blot, or FACS analysis). In some embodiments, Bcl-X_{L} expression is determined and compared to a reference. In some embodiments, Bcl-X_{L} expression in cells of a cancer is determined, relative to Bcl-X_{L} expression in cells of a non-cancerous tissue, e.g., a non-cancerous tissue of the same tissue type as the tumor. In some embodiments, Bcl-X_{L} expression in a lymphoma is determined, relative to Bcl-X_{L} expression in non-cancerous lymphocytes. In some embodiments, the percentage of Bcl-X_{L}⁺ or Bcl-X_{L}⁻ cells in a sample from a cancer are determined. Methods of analyzing and quantitating Bcl-X_{L} expression in patient samples, primary cells, and cell lines, are described, e.g., in Zhao et al., Blood 103:695-697, 2004; and Findley et al., Blood 89(8):2986-2993, 1997. In some embodiments, relative levels of Bcl-2 and Bcl-X_{L} expression are determined, e.g., to identify a subject whose cancer expresses more Bcl-2 than Bcl-X_{L}.

Treatment with romidepsin can involve selection and/or identification of subjects whose cancers are characterized by expression, or lack of expression, of other genes. In some embodiments, romidepsin treatment is indicated for a Bcl-2⁺ cancer that does not overexpress the multidrug transporter, P-glycoprotein (P-gp). P-gp is encoded by the MDR1 gene (Ueda et al., Proc. Natl. Acad. Sci. USA 84:3004, 1987). P-gp expression in cells of a cancer can be determined by any available means (e.g., using the MRK16 monoclonal antibody, or by detecting MDR1 mRNA expression).

As noted above, gene expression (e.g., Bcl-2 expression) can be determined by any available means. In some embodiments, a PCR-based method is used to analyze mRNA expression. In some embodiments, the method is RT-PCR. To perform RT-PCR, mRNA is isolated from a sample (e.g., total RNA isolated from a human lymphoma sample). mRNA can be extracted from a freshly isolated sample, from a frozen sample, or from an archived paraffin-embedded and fixed tissue sample. Methods for mRNA extraction are known in the art. See, e.g., Ausubel et al., Current Protocols in Molecular Biology, John Wiley and Sons, 1997. Methods for RNA extraction from paraffin embedded tissues are disclosed, for example, in Rupp and Locker, Lab Invest. 56:A67, 1987, and De Andres et al., BioTechniques 18:42044, 1995. Purification kits for RNA isolation from commercial manufacturers, such as Qiagen, can be used. For example, total RNA from a sample can be isolated using Qiagen RNeasy mini-columns, MasterPure™ Complete DNA and RNA Purification Kit (EPICENTRE™, Madison, Wis.), Paraffin Block RNA Isolation Kit (Ambion, Inc.), or RNA Stat-60 (Tel-Test) or other means. Next, RNA is reverse transcribed into cDNA, and the cDNA is amplified by PCR. Guidelines for PCR primer and probe design include, e.g., Dieffenbach et al., “General Concepts for PCR Primer Design” in: PCR Primer, A Laboratory Manual, Cold Spring Harbor Laboratory Press, New York, 133-155, 1995; Innis and Gelfand, “Optimization of PCRs” in: PCR Protocols, A Guide to Methods and Applications, CRC Press, London, 5-11, 1994; and Plasterer, T. N. Primerselect: Primer and probe design. Methods Mol. Biol. 70:520-527, 1997. Factors considered in PCR primer design include primer length, melting temperature (Tm), and G/C content, specificity, complementary primer sequences, and 3′-end sequence. PCR primers are generally 17-30 bases in length, with Tm's between 50-80° C.

In some embodiments, the PCR analysis is quantitative. In one embodiment of quantitative PCR, a third oligonucleotide, or probe, is used to detect nucleotide sequence located between the two PCR primers. The probe is non-extendible by the thermostable DNA polymerase used for PCR (e.g., Taq polymerase), and typically is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative analysis. RT-PCR can be performed using commercially available equipment, such as an ABI PRISM 7700™ Sequence Detection System (Perkin-Elmer-Applied Biosystems, Foster City, Calif., USA), or Lightcycler® (Roche Molecular Biochemicals, Mannheim, Germany). Samples can be analyzed using a real-time quantitative PCR device such as the ABI PRISM 7700™ Sequence Detection System™. To minimize errors and the effect of sample-to-sample variation, RT-PCR is usually performed using an internal standard. A suitable internal standard is expressed at a constant level among different tissues, and is unaffected by the experimental variable. RNAs frequently used to normalize patterns of gene expression are mRNAs for the housekeeping genes glyceraldehyde-3-phosphate-dehydrogenase (GAPDH) and β-actin.

A variation of the RT-PCR technique is real time quantitative PCR, which measures PCR product accumulation through a dual-labeled fluorogenic probe (i.e., TaqMan™ probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g., Held et al., Genome Res. 6:986-994, 1996. Methods for obtaining quantitative measures of gene expression are described, e.g., in WO 02/086498.

Another approach for gene expression analysis employs competitive PCR design and automated, high-throughput matrix-assisted laser desorption ionization time-of-flight (MALDI-TOF) MS detection and quantification of oligonucleotides (see Ding and Cantor, Proc. Natl. Acad. Sci. USA 100:3059-3064, 2003).

Additional PCR-based techniques for gene expression analysis include, e.g., differential display (Liang and Pardee, Science 257:967-971, 1992); amplified fragment length polymorphism (iAFLP) (Kawamoto et al., Genome Res. 12:1305-1312, 1999); BeadArray™ technology (Illumina, San Diego, Calif.; Oliphant et al., Discovery of Markers for Disease (Supplement to Biotechniques), June 2002; Ferguson et al., Anal. Chem. 72:5618, 2000); BeadsArray for Detection of Gene Expression (BADGE), using the commercially available Luminex100 LabMAP system and multiple color-coded microspheres (Luminex Corp., Austin, Tex.) in a rapid assay for gene expression (Yang et al., Genome Res. 11:1888-1898, 2001); and high coverage expression profiling (HiCEP) analysis (Fukumura et al., Nucl. Acids. Res. 31(16) e94, 2003).

Gene expression can also be analyzed by in situ hybridization, such as fluorescence in situ hybridization. See, e.g., Vogel et al., J. Clin. Oncol. 20(3):719-26, 2002, and Bartlett et al., J. Pathol., 199(4):411-7, 2003.

In some embodiments, gene expression is analyzed using a microarray. Typically, polynucleotides of interest are plated, or arrayed, on a microchip substrate. The arrayed sequences are then hybridized with nucleic acids (e.g., DNA or RNA) from cells or tissues of interest (e.g., lymphoma). The source of mRNA typically is total RNA (e.g., total RNA isolated from human lymphoma samples, and normal control samples). Probes are immobilized on an array substrate (e.g., a porous or nonporous solid support, such as a glass, plastic, or gel surface). The probes can include DNA, RNA, copolymer sequences of DNA and RNA, DNA and/or RNA analogues, or combinations thereof.

Microarrays can be addressable arrays, and more preferably positionally addressable arrays, i.e., each probe of the array is located at a known, predetermined position on the solid support such that the identity (i.e., the sequence) of each probe can be determined from its position in the array.

Each probe on the microarray can be between 10-50,000 nucleotides, e.g., between 300-1,000 nucleotides in length. The probes of the microarray can consist of nucleotide sequences with lengths less than 1,000 nucleotides, e.g., sequences 10-1,000, or 10-500, or 10-200 nucleotides in length. An array can include positive control probes, e.g., probes known to be complementary and hybridizable to sequences in the test sample, and negative control probes, e.g., probes known to not be complementary and hybridizable to sequences in the test sample.

Methods for attaching nucleic acids to a surface are known. See, e.g., Schena et al, Science 270:467-470, 1995; DeRisi et al, Nat. Genet. 14:457-460, 1996; Shalon et al., Genome Res. 6:639-645, 1996; and Schena et al., Proc. Natl. Acad. Sci. U.S.A. 93:10539-11286, 1995; U.S. Pat. Nos. 5,578,832; 5,556,752; 5,510,270; Maskos and Southern, Nuc. Acids. Res. 20:1679-1684, 1992. In principle, any type of array, for example, dot blots on a nylon hybridization membrane can be used (see Sambrook et al., Molecular Cloning, A Laboratory Manual, 2nd Ed., Vols. 1-3, Cold Spring Harbor Laboratory, Cold Spring Harbor, N.Y. (1989)).

Polynucleotide molecules to be analyzed may be from any clinically relevant source, and are expressed RNA or a nucleic acid derived therefrom (e.g., cDNA or amplified RNA derived from cDNA that incorporates an RNA polymerase promoter), including naturally occurring nucleic acid molecules, as well as synthetic nucleic acid molecules. For example, the test polynucleotide molecules include total cellular RNA, poly(A)+ messenger RNA (mRNA), or fraction thereof, cytoplasmic mRNA, or RNA transcribed from cDNA (i.e., cRNA; see, e.g., U.S. Pat. Nos. 5,545,522, 5,891,636, or 5,716,785). Nucleic acid hybridization and wash conditions are chosen so that the test polynucleotide molecules (e.g., polynucleotides from a lymphoma sample) specifically bind or specifically hybridize to the complementary polynucleotide sequences of the array, preferably to a specific array site, wherein its complementary nucleic acid is located. General parameters for specific (i.e., stringent) hybridization conditions for nucleic acids are described in Sambrook et al., supra, and in Ausubel et al., Current Protocols in Molecular Biology, vol. 2, Current Protocols Publishing, New York, 1994. Typically, stringent conditions for short probes (e.g., 10 to 50 nucleotide bases) will be those in which the salt concentration is at least about 0.01 to 1.0 M at pH 7.0 to 8.3 and the temperature is at least about 30° C. Stringent conditions can also be achieved with the addition of destabilizing agents such as formamide. When fluorescently labeled probes are used, the fluorescence emissions at each site of a microarray can be detected by scanning confocal laser microscopy or other methods (see Shalon et al., Genome Res. 6:639-645, 1996; Schena et al., Genome Res. 6:639-645, 1996; and Ferguson et al., Nat. Biotech. 14:1681-1684, 1996). Signals are recorded and typically analyzed by computer. Methods for evaluating microarray data and classifying samples are described in U.S. Pat. No. 7,171,311.

In some embodiments, gene expression is determined using a method that detects polypeptides (e.g., Bcl-2 polypeptides). Antibodies specific for a gene product of interest (e.g., Bcl-2, Bcl-X_{L}, P-gp) can be used to detect expression. Antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Exemplary immunoassays include, e.g., ELISA, radioimmunoassays, Western blot analysis, immunoprecipitation, immunohistochemical assays (see., e.g., Vogel et al., J. Clin. Oncol., 20(3):719-26, 2002, and Bartlett et al., J. Pathol., 199(4):411-7, 2003) Immunoassay protocols and kits are well known in the art and are commercially available.

In various aspects, the expression of certain genes in a sample from a cancer (e.g., a sample from a lymphoma) is detected to provide clinical information (e.g., classification of the cancer from which the sample is derived as a Bcl-2-oyerexpressing cancer). Thus, gene expression assays include measures to correct for differences in sample variability and quality. For example, an assay to detect mRNA typically measures and incorporates the mRNA expression of certain normalizing genes, such known housekeeping genes, e.g., GAPDH and β-actin. Alternatively, normalization can be based on a mean or median signal (Ct) of assayed genes or a large subset thereof (global normalization approach). In some embodiments, an amount of a gene expression product in a normalized test sample (e.g., from a patient sample) is compared to the amount found in a cancer sample, and/or normal sample reference set. The level of expression measured in a particular test sample can be determined to fall at some percentile within a range observed in reference sets.

### Romidepsin

The HDAC inhibitor romidepsin is used in accordance with the present invention for treating cancers identified as expressing, or lacking expression of, certain factors. For example, as described herein, romidepsin is used to treat Bcl-2⁺ lymphomas, Bcl-XL⁻ lymphomas, Bcl-2⁺ Bcl-XL⁻ lymphomas, or Bcl-2⁺ lymphomas that do not overexpress P-glycoprotein. Romidepsin is a cyclic depsipeptide of formula:

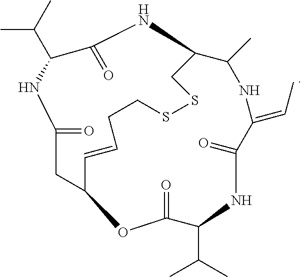

Romidepsin may be provided in any form. Pharmaceutically acceptable forms are particular preferred. Exemplary forms of romidepsin include, but are not limited to, salts, esters, pro-drugs, isomers, stereoisomers (e.g., enantiomers, diastereomers), tautomers, protected forms, reduced forms, oxidized forms, derivatives, and combinations thereof, with the desired activity (e.g., deacetylase inhibitory activity, aggresome inhibition, cytotoxicity). In certain embodiments, the romidepsin used in the combination therapy is pharmaceutical grade material and meets the standards of the U.S. Pharmacopoeia, Japanese Pharmacopoeia, or European Pharmacopoeia. In certain embodiments, the romidepsin is at least 95%, at least 98%, at least 99%, at least 99.9%, or at least 99.95% pure. In certain embodiments, the romidepsin is at least 95%, at least 98%, at least 99%, at least 99.9%, or at least 99.95% monomeric. In certain embodiments, no impurities are detectable in the romidepsin materials (e.g., oxidized material, reduced material, dimerized or oligomerized material, side products, etc.). The romidepsin typically includes less than 1.0%, less than 0.5%, less than 0.2%, or less than 0.1% of total other unknowns. The purity of romidepsin may be assessed by appearance, HPLC, specific rotation, NMR spectroscopy, IR spectroscopy, UV/Visible spectroscopy, powder x-ray diffraction (XRPD) analysis, elemental analysis, LC-mass spectroscopy, and mass spectroscopy.

The inventive therapy may also include a derivative of romidepsin. In certain embodiments, the derivative of romidepsin is of the formula (I):

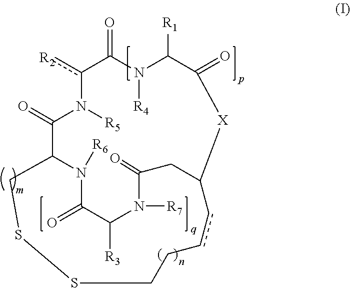

wherein

m is 1, 2, 3 or 4;

n is 0, 1, 2 or 3;

p and q are independently 1 or 2;

X is O, NH, or NR₈;

R₁, R₂, and R₃ are independently hydrogen; unsubstituted or substituted, branched or unbranched, cyclic or acyclic aliphatic; unsubstituted or substituted, branched or unbranched, cyclic or acyclic heteroaliphatic; unsubstituted or substituted aryl; or unsubstituted or substituted heteroaryl; and

R₄, R₅, R₆, R₇ and R₈ are independently hydrogen; or substituted or unsubstituted, branched or unbranched, cyclic or acyclic aliphatic; and pharmaceutically acceptable forms thereof. In certain embodiments, m is 1. In certain embodiments, n is 1. In certain embodiments, p is 1. In certain embodiments, q is 1. In certain embodiments, X is O. In certain embodiments, R₁, R₂, and R₃ are unsubstituted, or substituted, branched or unbranched, acyclic aliphatic. In certain embodiments, R₄, R₅, R₆, and R₇ are all hydrogen.

In certain embodiments, the derivative of romidepsin is of the formula (II):

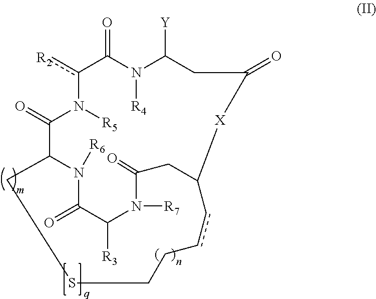

wherein:

m is 1, 2, 3 or 4;

n is 0, 1, 2 or 3;

q is 2 or 3;

X is O, NH, or NR₈;

Y is OR₈, or SR₈;

R₂ and R₃ are independently hydrogen; unsubstituted or substituted, branched or unbranched, cyclic or acyclic aliphatic; unsubstituted or substituted, branched or unbranched, cyclic or acylic heteroaliphatic; unsubstituted or substituted aryl; or unsubstituted or substituted heteroaryl;

R₄, R₅, R₆, R₇ and R₈ are independently selected from hydrogen; or substituted or unsubstituted, branched or unbranched, cyclic or acyclic aliphatic; and pharmaceutically acceptable forms thereof In certain embodiments, m is 1. In certain embodiments, n is 1. In certain embodiments, q is 2. In certain embodiments, X is O. In other embodiments, X is NH. In certain embodiments, R₂ and R₃ are unsubstituted or substituted, branched or unbranched, acyclic aliphatic. In certain embodiments, R₄, R₅, R₆, and R₂ are all hydrogen.

In certain embodiments, the derivative of romidepsin is of the formula (III):

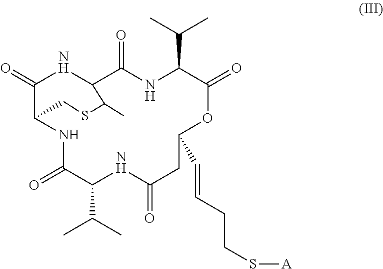

wherein

A is a moiety that is cleaved under physiological conditions to yield a thiol group and includes, for example, an aliphatic or aromatic acyl moiety (to form a thioester bond); an aliphatic or aromatic thioxy (to form a disulfide bond); or the like; and pharmaceutically acceptable forms thereof. Such aliphatic or aromatic groups can include a substituted or unsubstituted, branched or unbranched, cyclic or acyclic aliphatic group; a substituted or unsubstituted aromatic group; a substituted or unsubstituted heteroaromatic group; or a substituted or unsubstituted heterocyclic group. A can be, for example, —COR₁, —SC(═O)—O—R₁, or —SR₂. R₁ is independently hydrogen; substituted or unsubstituted amino; substituted or unsubstituted, branched or unbranched, cyclic or acyclic aliphatic; substituted or unsubstituted aromatic group; substituted or unsubstituted heteroaromatic group; or a substituted or unsubstituted heterocyclic group. In certain embodiment, R₁ is hydrogen, methyl, ethyl, n-propyl, iso-propyl, n-butyl, isobutyl, benzyl, or bromobenzyl. R₂ is a substituted or unsubstituted, branched or unbranched, cyclic or acyclic aliphatic group; a substituted or unsubstituted aromatic group; a substituted or unsubstituted heteroaromatic group; or a substituted or unsubstituted heterocyclic group. In certain embodiments, R₂ is methyl, ethyl, 2-hydroxyethyl, isobutyl, fatty acids, a substituted or unsubstituted benzyl, a substituted or unsubstituted aryl, cysteine, homocysteine, or glutathione.

In certain embodiments, the derivative of romidepsin is of formula (IV) or (IV′):

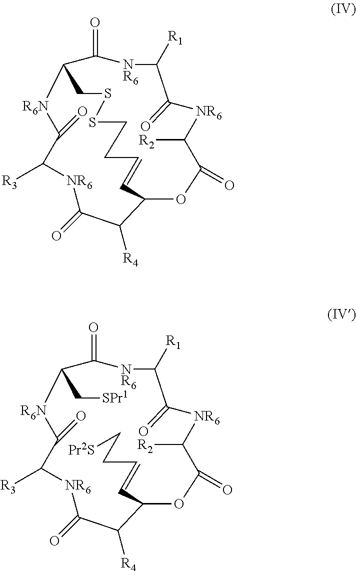

wherein

R₁, R₂, R₃, and R₄ are the same or different and represent an amino acid side chain moiety, each R₆ is the same or different and represents hydrogen or C₁-C₄ alkyl, and Pr¹ and Pr² are the same or different and represent hydrogen or thiol-protecting group. In certain embodiments, the amino acid side chain moieties are those derived from natural amino acids. In other embodiments, the amino acid side chain moieties are those derived from unnatural amino acids. In certain embodiments, each amino acid side chain is a moiety selected from —H, —C₁-C₆ alkyl, —C₂-C₆ alkenyl, -L-O—C(O)—R′, -L-C(O)—O—R″, -L-A, -L-NR″R″, -L-Het-C(O)-Het-R″, and -L-Het-R″, wherein L is a C₁-C₆ alkylene group, A is phenyl or a 5- or 6-membered heteroaryl group, each R′ is the same or different and represents C₁-C₄ alkyl, each R″ is the same or different and represent H or C₁-C₆ alkyl, each -Het- is the same or different and is a heteroatom spacer selected from —O—, —N(R′″)—, and —S—, and each R′″ is the same of different and represents H or C₁-C₄ alkyl. In certain embodiments, R₆ is —H. In certain embodiments, Pr¹ and Pr² are the same or different and are selected from hydrogen and a protecting group selected from a benzyl group which is optionally substituted by C₁-C₆ alkoxy, C₁-C₆ acyloxy, hydroxy, nitro, picolyl, picolyl-N-oxide, anthrylmethyl, diphenylmethyl, phenyl, t-butyl, adamanthyl, C₁-C₆ acyloxymethyl, C₁-C₆ alkoxymethyl, tetrahydropyranyl, benzylthiomethyl, phenylthiomethyl, thiazolidine, acetamidemethyl, benzamidomethyl, tertiary butoxycarbonyl (BOC), acetyl and its derivatives, benzoyl and its derivatives, carbamoyl, phenylcarbamoyl, and C₁-C₆ alkylcarbamoyl. In certain embodiments, Pr¹ and Pr² are hydrogen. Various romidepsin derivatives of formula (IV) and (IV′) are disclosed in published PCT application WO 2006/129105, published Dec. 7, 2006; which is incorporated herein by reference.

Processes for preparing romidepsin are known in the art. For example, exemplary processes of preparing romidepsin are described in U.S. Ser. No. 60/882,698, filed on Dec. 29, 2006; U.S. Ser. No. 60/882,704, filed on Dec. 29, 2006; and U.S. Ser. No. 60/882,712, filed on Dec. 29, 2006, the teachings of all of which are incorporated by reference herein. Since romidepsin is a natural product, it is typically prepared by isolating it from a fermentation of a microorganism that produces it. In certain embodiments, the romidepsin or a derivate thereof is purified from a fermentation, for example, of Chromobacterium violaceum. See, e.g., Ueda et al., J. Antibiot. (Tokyo) 47:301-310, 1994; Nakajima et al., Exp. Cell Res. 241:126-133, 1998; WO 02/20817; U.S. Pat. No. 4,977,138; each of which is incorporated herein by reference. In other embodiments, romidepsin or a derivative thereof is prepared by synthetic or semi-synthetic means. J. Am. Chem. Soc. 118:7237-7238, 1996; incorporated herein by reference.

The therapeutically effective amount of romidepsin will vary depending on the patient, the cancer being treated, stage of the cancer, pathology of the cancer, genotype of the cancer, phenotype of the cancer, the route of administration, etc. In certain embodiments, the romidepsin is dosed in the range of 0.5 mg/m² to 32 mg/m². In certain embodiments, the romidepsin is dosed in the range of 0.5 mg/m² to 28 mg/m². In certain embodiments, the romidepsin is dosed in the range of 1 mg/m² to 25 mg/m². In certain embodiments, the romidepsin is dosed in the range of 0.5 mg/m² to 15 mg/m². In certain embodiments, the romidepsin is dosed in the range of 1 mg/m² to 15 mg/m². In certain embodiments, the romidepsin is dosed in the range of 1 mg/m² to 8 mg/m². In certain embodiments, the romidepsin is dosed in the range of 0.5 mg/m² to 5 mg/m². In certain embodiments, the romidepsin is dosed in the range of 2 mg/m² to 10 mg/m². In certain embodiments, the romidepsin is dosed in the range of 4 mg/m² to 15 mg/m². In certain embodiments, the romidepsin is dosed in the range of 8 mg/m² to 10 mg/m². In other embodiments, the dosage ranges from 10 mg/m² to 20 mg/m². In certain embodiments, the dosage ranges from 5 mg/m² to 10 mg/m². In other embodiments, the dosage ranges from 10 mg/m² to 15 mg/m². In still other embodiments, the dosage is approximately 8 mg/m². In still other embodiments, the dosage is approximately 9 mg/m². In still other embodiments, the dosage is approximately 10 mg/m². In still other embodiments, the dosage is approximately 11 mg/m². In still other embodiments, the dosage is approximately 12 mg/m². In still other embodiments, the dosage is approximately 13 mg/m². In still other embodiments, the dosage is approximately 14 mg/m². In still other embodiments, the dosage is approximately 15 mg/m². In certain embodiments, increasing doses of romidepsin are administered over the course of a cycle. For example, in certain embodiments, a dose of approximately 8 mg/m², followed by a dose of approximately 10 mg/m², followed by a dose of approximately 12 mg/m² may be administered over a cycle. As will be appreciated by one of skill in the art, depending on the form of romidepsin being administered the dosing may vary. The dosages given herein are dose equivalents with respect to the active ingredient, romidepsin. As will be appreciated by one of skill in the art, more of a salt, hydrate, co-crystal, pro-drug, ester, solute, etc. may need to be administered to deliver the equivalent number of molecules of romidepsin. In certain embodiments, romidepsin is administered intravenously. In certain embodiments, the romidepsin is administered intravenously over a 1-6 hour time frame. In certain particular embodiments, the romidepsin is administered intravenously over 3-4 hours. In certain particular embodiments, the romidepsin is administered intravenously over 5-6 hours. In certain embodiments, the romidepsin is administered one day followed by several days in which the romidepsin is not administered.

In some embodiments, a patient receives a higher dose and/or longer course of treatment based on Bcl-2 expression of the patient's tumor. For example, in some embodiments, a patient with a lymphoma that overexpresses Bcl-2 is administered a higher dose of romidepsin than would be administered to a patient with a lymphoma that does not overexpress Bcl-2 (e.g., a patient with a lymphoma that overexpresses Bcl-2 is administered a dose at the high range of doses normally given to a patient of the same weight).

In certain embodiments, romidepsin is administered in an accelerated dosing regimen, e.g., such that one or more individual doses is administered over a period of time that is less than about 50 minutes, 40 minutes, 30 minutes, 20 minutes, or less. In some embodiments of an accelerated dosing regimen, one or more doses of romidepsin are administered intravenously. In some embodiments of an accelerated dosing regimen, one or more doses of romidepsin are administered by a route other than intravenous administration (e.g., oral, subcutaneous, nasal, topical, etc.).

In certain embodiments, romidepsin and a second anti-neoplastic agent are administered together. In other embodiments, the romidepsin and a second anti-neoplastic agent are administered separately. For example, the administration of romidepsin and a second agent may be separated by one or more days.

In certain embodiments, romidepsin is administered twice a week. In certain embodiments, romidepsin is administered once a week. In other embodiments, romidepsin is administered every other week. In certain embodiments, romidepsin is administered on days 1, 8, and 15 of a 28 day cycle. In certain particular embodiments, an 8 mg/m² dose of romidepsin is administered on day 1, a 10 mg/m² dose of romidepsin is administered on day 8, and a 12 mg/m² dose of romidepsin is administered on day 15. In certain embodiments, romidepsin is administered on days 1 and 15 of a 28 day cycle. The 28 day cycle may be repeated. In certain embodiments, the 28 day cycle is repeated 3-10 times. In certain embodiments, the treatment includes 5 cycles. In certain embodiments, the treatment includes 6 cycles. In certain embodiments, the treatment includes 7 cycles. In certain embodiments, the treatment includes 8 cycles. In certain embodiments, greater than 10 cycles are administered. In certain embodiments, the cycles are continued as long as the patient is responding. The therapy may be terminated once there is disease progression, a cure or remission is achieved, or side effects become intolerable.

To give but a few examples of appropriate dosing schedules for use in accordance with the present invention, romidepsin may be administered daily (for example for 2 weeks), twice weekly (for example for 4 weeks), thrice weekly (for example for 4 weeks), or on any of a variety of other intermittent schedules (e.g., on days 1, 3, and 5; on days 4 and 10; on days 1 and 15; on days 5 and 12; or on days 5, 12, and 19 of 21 or 28 day cycles).

In certain embodiments, romidepsin is administered on days 1, 8, and 15 of a 28 day cycle. In certain particular embodiments, an 8 mg/m² dose of romidepsin is administered on day 1, a 10 mg/m² dose of romidepsin is administered on day 8, and a 12 mg/m² dose of romidepsin is administered on day 15. In certain embodiments, romidepsin is administered on days 1 and 15 of a 28 day cycle with day 8 being skipped. A 28 day dosing cycle may be repeated. In certain embodiments, a 28 day cycle is repeated 2-10, 2-7, 2-5, or 3-10 times. In certain embodiments, the treatment includes 5 cycles. In certain embodiments, the treatment includes 6 cycles. In certain embodiments, the treatment includes 7 cycles. In certain embodiments, the treatment includes 8 cycles. In certain embodiments, 10 cycles are administered. In certain embodiments, greater than 10 cycles are administered.

In some embodiments, romidepsin is administered orally. In certain embodiments, romidepsin is dosed orally in the range of 10 mg/m² to 300 mg/m². In certain embodiments, romidepsin is dosed orally in the range of 25 mg/m² to 100 mg/m². In certain embodiments, romidepsin is dosed orally in the range of 100 mg/m² to 200 mg/m². In certain embodiments, romidepsin is dosed orally in the range of 200 mg/m² to 300 mg/m². In certain embodiments, romidepsin is dosed orally at greater than 300 mg/m². In certain embodiments, romidepsin is dosed orally in the range of 50 mg/m² to 150 mg/m². In other embodiments, the oral dosage ranges from 25 mg/m² to 75 mg/m². As will be appreciated by one of skill in the art, depending on the form of romidepsin being administered the dosing may vary. The dosages given herein are dose equivalents with respect to the active ingredient, romidepsin. In certain embodiments, romidepsin is administered orally on a daily basis. In other embodiments, romidepsin is administered orally every other day. In still other embodiments, romidepsin is administered orally every third, fourth, fifth, or sixth day. In certain embodiments, romidepsin is administered orally every week. In certain embodiments, romidepsin is administered orally every other week. In certain embodiments, romidepsin and a second anti-neoplastic agent are administered together. In other embodiments, romidepsin and the second agent are administered separately. For example, the administration of romidepsin and a second agent may be separated by one or more days. In certain embodiments, both romidepsin and the second agent are administered orally. In certain embodiments, only romidepsin is administered orally. The administration of romidepsin alone or the combination of romidepsin and the second agent may be terminated once there is disease progression, a cure or remission is achieved, or side effects become intolerable.

### Other Anti-Neoplastic Agents

Anti-neoplastic agents suitable for the present invention includes any agents that inhibit or prevent the growth of neoplasms, checking the maturation and proliferation of malignant cells. Growth inhibition can occur through the induction of stasis or cell death in the tumor cell(s). Typically, antineoplastic agents include cytotoxic agents in general. Exemplary anti-neoplastic agents include, but are not limited to, cytokines, ligands, antibodies, radionuclides, proteasome inhibitors, kinase inhibitors, mitotic inhibitors, nucleoside analogs, alkylating agents, antimetabolites, and other types of chemotherapeutic agents. In particular, such agents include bortezomib (VELCADE®), interleukin 2 (IL-2), interferon (IFN) TNF; photosensitizers, including aluminum (III) phthalocyanine tetrasulfonate, hematoporphyrin, and phthalocyanine; radionuclides, such as iodine-131 (¹³¹I), yttrium-90 (⁹⁰Y), bismuth-212 (²¹²Bi), bismuth-213 (²¹³Bi), technetium-99m (.^{99m}Tc), rhenium-186 (¹⁸⁶Re), and rhenium-188 (¹⁸⁸Re); chemotherapeutics, such as neocarzinostatin, bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from chinese cobra (naja naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; ly207702 (a difluorinated purine nucleoside); liposomes containing antitumor agents (e.g., antisense oligonucleotides, plasmids encoding toxins, methotrexate, etc.); and antibodies or antibody fragments, such as F(ab).

In certain embodiments, romidepsin is administered in combination with an alkylating agent. Exemplary alkylating agents include nitrogen mustards (e.g., mechlorethamine, cyclophosphamide, ifosfamide, melphalan,and chlorambucil), ethylenimines and methylmelamines (e.g., hexamethylmelamine, thiotepa), alkyl sulfonates (e.g., busulfan), nitrosoureas (e.g., carmustine, lomustine, semustine, streptozocin), and triazenes (e.g., dacarbazine (dimethyltriazenoimid-azolecarboxamide)).

In certain embodiments, romidepsin is administered in combination with an antimetabolite. Exemplary antimetabolites include folic acid analogs (e.g., methotrexate), pyrimidine analogs (e.g., fluorouracil, cytarabine), and purine analogs (e.g., fludarabine, idarubicin, cytosine arabinoside, mercaptopurine, thioguanine, pentostatin). Other examples of anti-neoplastic agents that can be administered in combination with romidepsin include vinca alkaloids (e.g., vinblastine, vincristine, vendesine), epipodophyllotoxins (e.g., etoposide, teniposide), antibiotics (e.g., dactinomycin, daunorubicin, doxorubicin, bleomycin, plicamycin and mitomycin), dibromomannitol, deoxyspergualine, enzymes (e.g., L-asparaginase), biological response modifiers such as interferon-alpha, platinum coordination complexes (e.g., cisplatin, carboplatin), substituted urea (e.g., hyroxyurea), anthracenedione (e.g., mitoxantrone), and methylhydrazine derivatives (e.g., procarbazine), adrenocortical suppressants (e.g., mitotane, aminoglutethimide).

In certain embodiments, romidepsin is administered in combination with a steroidal agent. Exemplary steroidal agents suitable for the present invention include, but are not limited to, alclometasone diproprionate, amcinonide, beclomethasone diproprionate, betamethasone, betamethasone benzoate, betamethasone diproprionate, betamethasone sodium phosphate, betamethasone sodium phosphate and acetate, betamethasone valerate, clobetasol proprionate, clocortolone pivalate, cortisol (hydrocortisone), cortisol (hydrocortisone) acetate, cortisol (hydrocortisone) butyrate, cortisol (hydrocortisone) cypionate, cortisol (hydrocortisone) sodium phosphate, cortisol (hydrocortisone) sodium succinate, cortisol (hydrocortisone) valerate, cortisone acetate, desonide, desoximetasone, dexamethasone, dexamethasone acetate, dexamethasone sodium phosphate, diflorasone diacetate, fludrocortisone acetate, flunisolide, fluocinolone acetonide, fluocinonide, fluorometholone, flurandrenolide, halcinonide, medrysone, methylprednisolone, methylprednisolone acetate, methylprednisolone sodium succinate, mometasone furoate, paramethasone acetate, prednisolone, prednisolone acetate, prednisolone sodium phosphate, prednisolone tebutate, prednisone, triamcinolone, triamcinolone acetonide, triamcinolone diacetate, and triamcinolone hexacetonide or a synthetic analog thereof, or a combination thereof. In certain embodiments, the steroidal agent suitable for the invention is dexamethasone. In certain embodiments, the steroidal agent suitable for the invention is prednisolone.

In certain embodiments, the steroidal agent is administered at a dosage ranging from 0.25 mg to 100 mg. In certain embodiments, the steroidal agent is administered at a dosage ranging from 5 mg to 60 mg. In certain embodiments, the steroidal agent is administered at a dosage ranging from 10 mg to 50 mg. In a particular embodiment, the steroidal agent is administered at a dosage of approximately 40 mg. In a particular embodiment, the steroidal agent is administered at a dosage of approximately 30 mg. In another particular embodiment, the steroidal agent is administered at a dosage of approximately 20 mg. In a particular embodiment, the steroidal agent is administered at a dosage of approximately 10 mg. In a particular embodiment, the steroidal agent is administered at a dosage of approximately 5 mg. In certain embodiments, the steroidal agent is administered concurrently with the romidepsin. In certain embodiments, the steroidal agent is administered prior to or following the administration of romidepsin. For example, the steroidal agent may be administered 5 to 7 days prior to the administration of romidepsin. In certain embodiments, the steroidal agent is dexamethasone, and the dosage of dexamethasone if 20 mg.

In certain embodiments, romidepsin is administered in combination with a proteasome inhibitor. Exemplary proteasome inhibitors include bortezomib (VELCADE®), peptide boronates, salinosporamide A (NPI-0052), lactacystin, epoxomicin (Ac(Me)-Ile-Ile-Thr-Leu-EX), MG-132 (Z-Leu-Leu-Leu-al), PR-171, PS-519, eponemycin, aclacinomycin A, CEP-1612, CVT-63417, PS-341 (pyrazylcarbonyl-Phe-Leu-boronate), PSI (Z-Ile-Glu(OtBu)-Ala-Leu-al), MG-262 (Z-Leu-Leu-Leu-bor), PS-273 (MNLB), omuralide (clasto-lactacystin-3-lactone), NLVS (Nip-Leu-Leu-Leu-vinyl sulfone), YLVS (Tyr-Leu-Leu-Leu-vs), dihydroeponemycin, DFLB (dansyl-Phe-Leu-boronate), ALLN (Ac-Leu-Leu-Nle-al), 3,4-dichloroisocoumarin, 4-(2-aminoethyl)-benzenesulfonyl fluoride, TMC-95A, gliotoxin, EGCG ((−)-epigallocatechin-3-gallate), and YU101 (Ac-hFLFL-ex). In certain embodiments, romidepsin is combined with bortezomib (VELCADE®).

In certain embodiments, romidepsin is administered in combination with a kinase inhibitor, e.g., a tyrosine kinase inhibitor. Tyrosine kinase inhibitors are agents that reduce the activity and/or amount of a tyrosine kinase in a cell. Such agents can be useful in combination with romidepsin the treatment of cancers as described herein (e.g., Bcl-2⁺ lymphomas). Commercially available tyrosine kinase inhibitors include, for example, axitinib, cediranib (RECENTIN), dasatinib (SPRYLCEL), erlotinib (TARCEVA®), gefitinib (IRESSA), imatinib (GLEEVEC), lapatinib, lestaurtinib, nilotinib, semaxanib, sunitinib, and vandetanib. In certain embodiments, romidepsin is used in combination with axitinib. In certain embodiments, romidepsin is used in combination with cediranib. In certain embodiments, romidepsin is used in combination with dasatinib. In certain embodiments, romidepsin is used in combination with erlotinib. Erlotinib specifically targets the epidermal growth factor receptor tyrosine kinase, which is highly expressed and occasionally mutated in various forms of cancer. In certain embodiments, romidepsin is used in combination with gefitinib. In certain embodiments, romidepsin is used in combination with imatinib. In certain embodiments, romidepsin is used in combination with lapatinib. In certain embodiments, romidepsin is used in combination with lestaurtinib. In certain embodiments, romidepsin is used in combination with nilotinib. In certain embodiments, romidepsin is used in combination with semaxanib. In certain embodiments, romidepsin is used in combination with sunitinib. In certain embodiments, romidepsin is used in combination with vandetanib. Other kinase inhibitors that may be used in combination with romidepsin include flavopiridol, LY294002, PKC412, and PD184352.

In cetain embodiments, romidepsin is administered with 17-allyl-amino-demethoxygeldanamycin (17-AAG).

In certain embodiments, romidepsin is administered with an agent that inhibits expression or activity of Bcl-X_{L}. Examples of such agents include antisense agents (see, e.g., U.S. Pat. No. 5,776,905 and U.S. Pat. Pub. No. 20030191300), and small molecules (see, e.g., WO02097053, U.S. Pat. Pub. No. 20030199489, and U. S. Pat. Pub. No. 20080057098).

In certain embodiments, romidepsin is administered in combination with an anti-mitotic agent (e.g., docetaxel, paclitaxel, or an epothilone such as epothilone B).

In certain embodiments, romidepsin is administered in combination with one or more cytotoxic agents. Exemplary such cytotoxic agents include, for example, gemcitabine, decitabine, and flavopiridol.

In certain embodiments, romidepsin is administered in combination with one or more anti-folates. For example, in some such embodiments, romidepsin is administered in combination with one or more of: folinic acid (leucovorin), methotrexate, pralatrexate, premextred, triazinate, and combinations thereof.

In certain embodiments, romidepsin is administered in combination with one or more methyl transferase inhibitors or demethylating agents (e.g., cytidine analogs such as 5-aza-2′-deoxycytidine, 5-azacytidine, and zebularine (1-[β-D-ribofuranosyl]-1,2-dihydropyrimidin-2-1).

In certain embodiments, romidepsin is administered in combination with one or more therapeutic antibodies. For example, in some such embodiments, romidepsin is administered in combination with one or more of: bevacizumab, cetuximab, dasatinib, erlotinib, geftinib, imatinib, lapatinib, nilotinib, panitumumab, pegaptanib, ranibizumab, sorafenib, sunitinib, trastuzumab, rituximab, or any antibody that binds to an antigen bound by one of these.

In certain embodiments, romidepsin is administered in conjunction with CHOP chemotherapy, i.e., therapy with cyclophosphamide, adriamycin (or doxorubicin), vincristine, and prednisolone (see, e.g., Coiffier et al., New Eng. J. Med. 346(4):235-42, 2002), or a subset of this combination.

In some embodiments, romidepsin is administered in combination with an anti-inflammatory agent such as aspirin, ibuprofen, acetaminophen, etc., pain reliever, anti-nausea medication, or anti-pyretic.

In certain embodiments, romidepsin is administered in combination with an agent to treat gastrointestinal disturbances such as nausea, vomiting, and diarrhea. Such agents may include anti-emetics, anti-diarrheals, fluid replacement, electrolyte replacement, etc.

In some embodiments, romidepsin is administered in combination with electrolyte replacement or supplementation such as potassium, magnesium, and calcium, in particular, potassium and magnesium (see below).

In certain embodiments, romidepsin is administered in combination with an anti-arrhythmic agent.

In certain embodiments, romidepsin is administered in combination with a platelet booster, for example, an agent that increases the production of platelets.

In certain embodiments, romidepsin is administered in combination with an agent to boost the production of blood cells such as erythropoietin.

In some embodiments, romidepsin is administered in combination with an agent to prevent hyperglycemia.

In certain embodiments, romidepsin is not administered with another HDAC or DAC inhibitor, e.g., an HDAC inhibitor which is a short chain fatty acid (e.g., butyrate, valproic acid, AN-9), or a hydroxyamate (e.g., trichostatin A, vorinostat (suberoylanilide hydroxyamic acid), PXD1, oxamflatin, LAQ824, LBH589, m-caroboxycinnamic acid bis-hydroxyamide, Scriptaid, pyroxyamide, suberic bishyroxyamic acid, azelaic bixhydroxyamic acid, SK-7041, SK-7068, CG-1521, Tubacin), or a benzamide (e.g., MS-275, CI-994), or a cyclic tetrapeptide (e.g., Trapoxin A, Apicidin, CHAPs), or an electrophilic ketone (e.g., trifluoromethoxyketone), or Depucidin, or MGCD-0103.

### Uses

Romidepsin may be used in vitro or in vivo. Romidepsin is particularly useful in the treatment of cancers, e.g., lymphomas, e.g., Bcl-2⁺ lymphomas, in vivo. However, romidepsin may also be used in vitro for research or clinical purposes (e.g., determining the susceptibility of a patient's disease to treatment with romidepsin, researching the mechanism of action, elucidating a cellular pathway or process).

Hematological malignancies are types of cancers that affect the blood, bone marrow, and/or lymph nodes. In certain embodiments, the malignancy is a Bcl-2⁺ hematological malignancy. In certain embodiments, the hematologic malignancy does not overexpress Bcl-X_{L}. In certain embodiments, the hematologic malignancy does not overexpress P-glycoprotein. In certain embodiments, the cancer is a lymphoma. In some embodiments, the cancer is a cutaneous T-cell lymphoma. In other embodiments, the cancer is peripheral T-cell lymphoma. In certain embodiments, the cancer is a Hodgkin's lymphoma, a non-Hodgkin's lymphoma, a follicular lymphoma, a B cell lymphoma, a diffuse large B cell lymphoma, a mantle cell lymphoma, or a Burkitt's lymphoma.

Other types of hematological malignancies, characterized by one or more of: Bcl-2 expression, lack of overexpression of Bcl-X_{L}, lack of overexpression of P-glycoprotein, and that may be treated include, but are not limited to: acute lymphoblastic leukemia (ALL), acute myelogenous leukemia (AML), chronic myelogenous leukemia (CML), chronic lymphocytic leukemia (CLL), hairy cell leukemia, and multiple myeloma. In certain embodiments, romidepsin is used to treat multiple myeloma. In certain particular embodiments, the cancer is relapsed and/or refractory multiple myeloma. In other embodiments, romidepsin is used to treat chromic lymphocytic leukemia (CLL). In certain embodiments, romidepsin is used to treat acute lymphoblastic leukemia (ALL). In certain embodiments, romidepsin is used to treat acute myelogenous leukemia (AML). In some embodiments, a method of treatment includes identifying the hematological malignancy as one which is characterized by one or more of: Bcl-2 expression, lack of overexpression of Bcl-X_{L}, lack of overexpression of P-glycoprotein, e.g., by evaluating gene expression as described herein.

Other cancers besides hematological malignancies may also be treated. In certain embodiments, the cancer is a solid tumor characterized by one or more of: Bcl-2 expression, lack of overexpression of Bcl-X_{L}, lack of overexpression of P-glycoprotein. Exemplary cancers that may be treated include colon cancer, lung cancer, bone cancer, pancreatic cancer, stomach cancer, esophageal cancer, skin cancer, brain cancer, liver cancer, ovarian cancer, cervical cancer, uterine cancer, testicular cancer, prostate cancer, bladder cancer, kidney cancer, neuroendocrine cancer, etc. In certain embodiments, romidepsin is used to treat pancreatic cancer. In certain embodiments, romidepsin is used to treat prostate cancer. In certain specific embodiments, the prostate cancer is hormone refractory prostate cancer. In some embodiments, a method of treatment includes identifying the solid tumor as one which is characterized by one or more of: Bcl-2 expression, lack of overexpression of Bcl-X_{L}, lack of overexpression of P-glycoprotein, e.g., by evaluating gene expression as described herein.

Romidepsin may also be used to treated a refractory or relapsed malignancy, e.g., a refractory or relapsed malignancy characterized by one or more of: Bcl-2 expression, lack of overexpression of Bcl-X_{L}, lack of overexpression of P-glycoprotein. In certain embodiments, the cancer is a refractory and/or relapsed hematological malignancy. For example, the cancer may be resistant to a particular chemotherapeutic agent. In certain embodiments, the cancer is a bortezomib-resistant malignancy. In other embodiments, the cancer is resistant to steroid therapy. In certain embodiments, the cancer is a hematological malignancy that is resistant steroid treatment. In certain embodiments, the cancer is steroid-resistant lymphoma. In certain particular embodiments, the cancer is dexamethasone-resistant lymphoma. In certain particular embodiments, the cancer is prednisolone-resistant lymphoma. In some embodiments, a method of treatment includes identifying the refractory or relapsed malignancy as one which is characterized by one or more of: Bcl-2 expression, lack of overexpression of Bcl-X_{L}, lack of overexpression of P-glycoprotein, e.g., by evaluating gene expression as described herein.

Romidepsin may also be used to treat and/or kill cells (e.g., Bcl-2⁺ cells) in vitro. A method of treatment in vitro can include identifying cells, prior to treatment, as cells which are characterized by one or more of: Bcl-2 expression, lack of overexpression of Bcl-X_{L}, lack of overexpression of P-glycoprotein, e.g., by evaluating gene expression as described herein. In some embodiments, expression of one or more of these factors is also evaluated during or after treatment. In certain embodiments, a cytotoxic concentration of romidepsin is contacted with the cells in order to kill them. In other embodiments, a sublethal concentration of romidepsin is used to treat the cells. In certain embodiments, the concentration of romidepsin ranges from 0.01 nM to 100 nM. In certain embodiments, the concentration of romidepsin ranges from 0.1 nM to 50 nM. In certain embodiments, the concentration of romidepsin ranges from 1 nM to 10 nM.

In certain embodiments, the cells are vertebrate cells. In certain embodiments, the cells are mammalian cells. In certain embodiments, the cells are human cells. The cells may be derived from a male or female human in any stage of development. In certain embodiments, the cells are primate cells. In other embodiments, the cells are derived from a rodent (e.g., mouse, rat, guinea pig, hamster, gerbil). In certain embodiments, the cells are derived from a domesticated animal such as a dog, cat, cow, goat, pig, etc. The cells may also be derived from a genetically engineered animal or plant, such as a transgenic mouse.

The cells used may be wild type or mutant cells. The cells may be genetically engineered (e.g., engineered to overexpress Bcl-2). In certain embodiments, the cells are normal cells. In certain embodiments, the cells are hematological cells. In certain embodiments, the cells are white blood cells. In certain embodiments, the white blood cells are lymphocytes (e.g., T cells or B cells). In certain embodiments, the white blood cells are myeloid cells (e.g., macrophages or monocytes). In certain particular embodiments, the cells are precursors of white blood cells (e.g., stem cells, progenitor cells, blast cells). In certain embodiments, the cells are neoplastic cells. In certain embodiments, the cells are cancer cells. In certain embodiments, the cells are derived from a hematological malignancy, e.g., a lymphoma, such as a cutaneous T cell lymphoma. In other embodiments, the cells are derived from a solid tumor. For example, the cells may be derived from a patient's tumor (e.g., from a biopsy or surgical excision). In certain embodiments, the cells are derived from a blood sample from the subject or from a bone marrow biopsy. In certain embodiments, the cells are derived from a lymph node biopsy. Such testing for cytotoxicity may be useful in determining whether a patient will respond to romidepsin therapy. Such testing may also be useful in determining the dosage needed to treat the malignancy. This testing of the susceptibility of a patient's cancer to the combination therapy would prevent the unnecessary administration of drugs with no effect to the patient. The testing may also allow the use of lower doses if the patient's cancer is particularly susceptible to romidepsin.

In other embodiments, the cells are derived from cancer cells lines. In certain embodiments, the cells are from hematological malignancies, e.g., Bcl-2⁺ lymphomas, such as those discussed herein. Human leukemia cell lines include U937, HL-60, THP-1, Raji, CCRF-CEM, and Jurkat. Exemplary CLL cell lines include JVM-3 and MEC-2. Exemplary myeloma cells lines include MM1.S, MM1.R (dexamethasone-resistant), RPMI8226, NCI-H929, and U266. Exemplary lymphoma cell lines includes Karpas, SUDH-6, SUDH-16, L428, KMH2, and Granta mantle lymphoma cell line. In certain embodiments, the cells are AML cells or multiple myeloma (CD138⁺) cells. In certain embodiments, the cells are hematopoietic stem or progenitor cells. For example, in certain embodiments, the cells are hematopoietic progenitor cells such as CD34⁺ bone marrow cells. In certain embodiments, the cell lines are resistant to a particular chemotherapeutic agent. In other embodiments, the cell line is steroid-resistant (e.g., dexamethasone-resistant, prednisolone-resistant).

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Romidepsin Can Overcome the Antiapoptotic Effects of Bcl-2 In Vitro

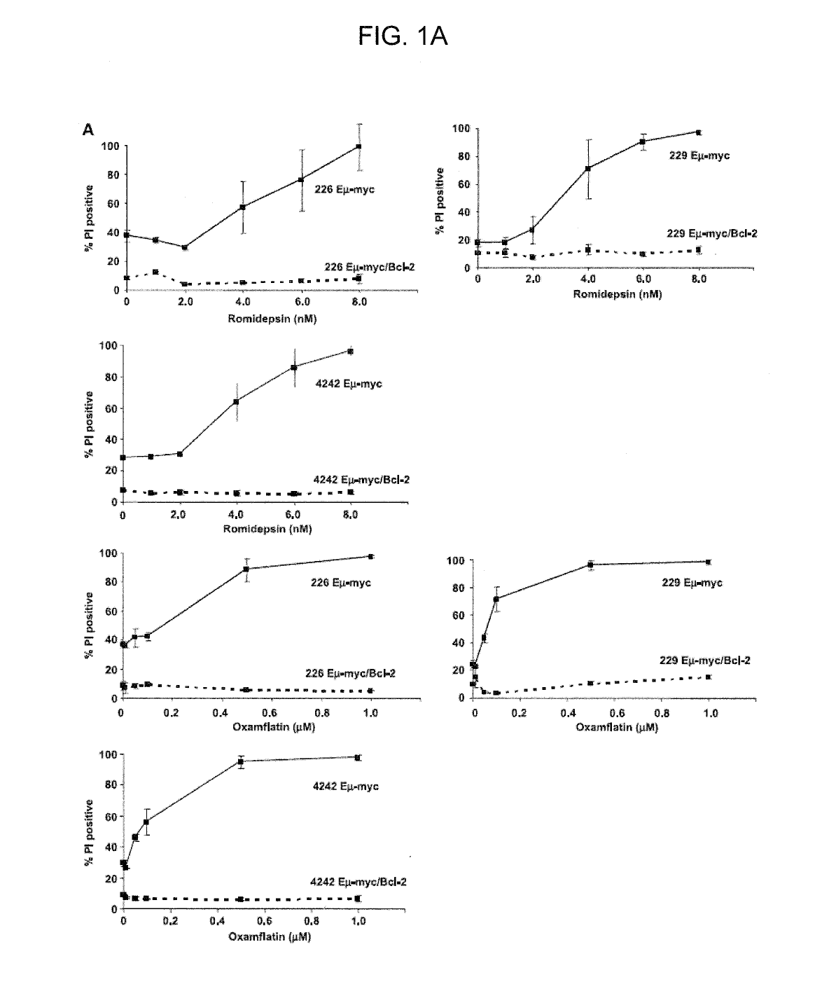

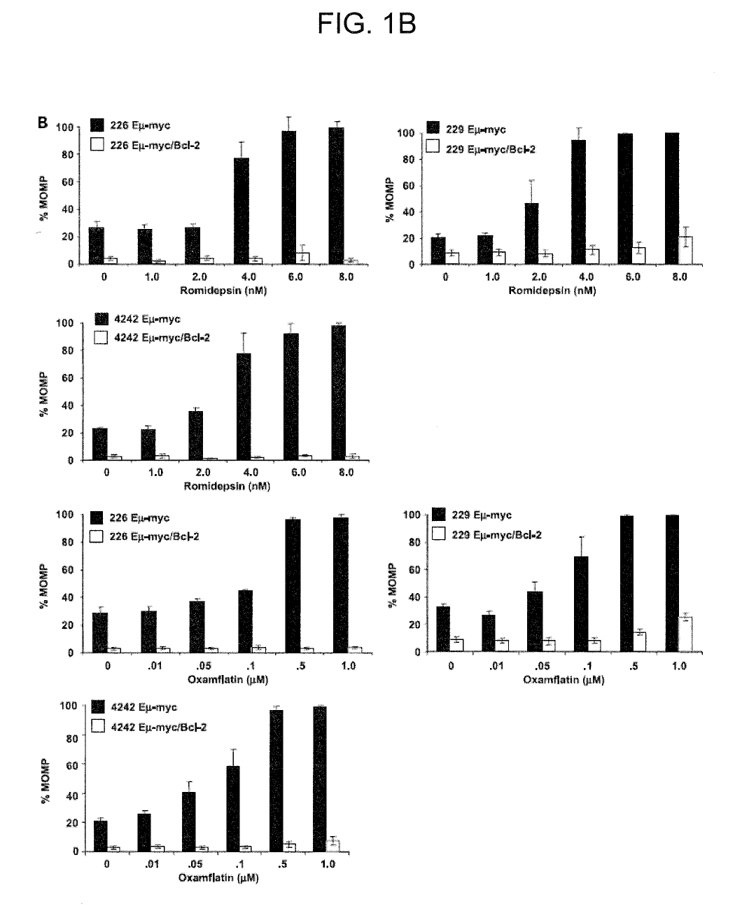

Three different primary Eμ-myc lymphomas overexpressing Bcl-2 and control vector-transduced Eμ-myc cells were tested for sensitivity to the histone deacetylase inhibitors (HDACi) oxamflatin and romidepsin. Both agents could effectively kill Eμ-myc but not Eμ-myc/Bcl-2 lymphomas in a 24-h dose-response assay as assessed by outer cell membrane damage (FIG. 1A-F) and loss of mitochondrial membrane potential (FIG. 1B).

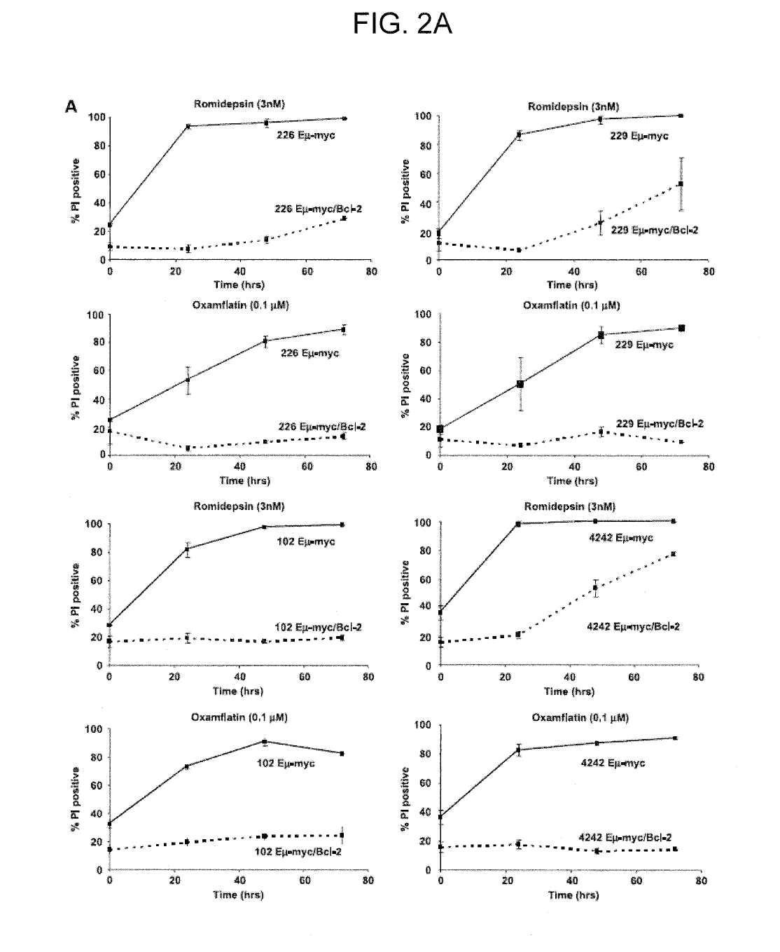

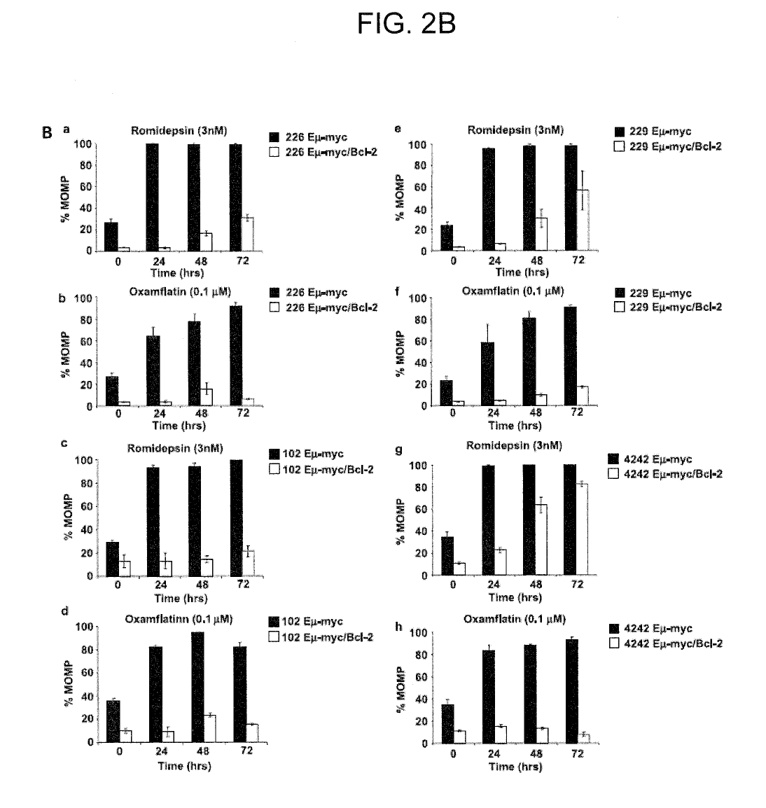

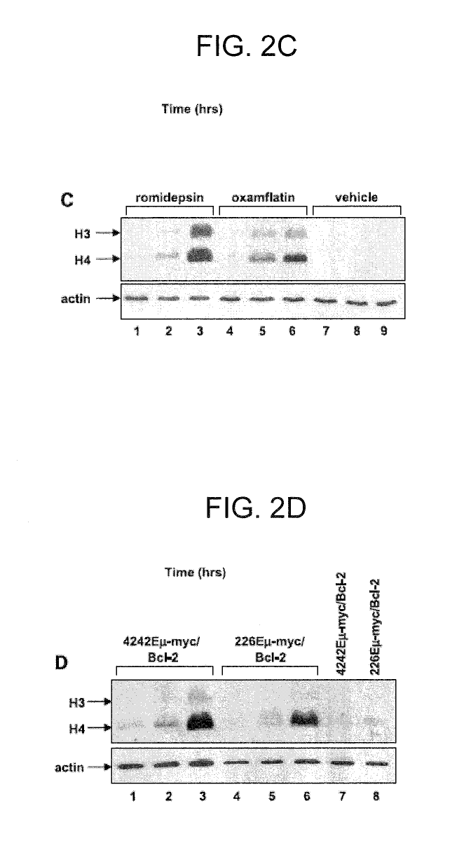

To determine if the HDACi-inhibitory effects of Bcl-2 were long lasting, a time course experiment was performed using doses of oxamflatin (0.1 μmol/L) and romidepsin (3.0 nmol/L) that were sufficient to kill Eμ-myc lymphomas in 24 h. Overexpression of Bcl-2 conferred resistance to oxamflatin even following 72 h of continuous exposure of the cells to this HDACi (FIG. 2A). In contrast, romidepsin could kill two of the four Eμ-myc/Bcl-2 lymphomas (4242Eμ-myc/Bcl-2 and 229Eμ-myc/Bcl-2) over time, whereas another two independently derived Eμ-myc/Bcl-2 lymphomas (102Eμ-myc/Bcl-2 and 226Eμ-myc/Bcl-2) remained relatively insensitive to romidepsin.

The primary function of prosurvival Bcl-2 proteins is to inhibit the activity of Bak and Bax proteins and thereby protect the mitochondrial membrane from damage (Cory et al., Nat. Rev. Cancer 2:647-656, 2002). To determine if the induction of apoptosis mediated by romidepsin in 4242Eμ-myc/Bcl-2 and 229Eμ-myc/Bcl-2 lymphomas was via perturbation of the mitochondrial membrane or through some other mechanism, HDACi-induced mitochondrial outer membrane permeabilization (MOMP) was quantitated by staining with tetramethylrhodamine ethyl ester (Molecular Probes). Consistent with the data shown in FIG. 2A, oxamflatin and romidepsin induced robust MOMP in all four Eμ-myc/MSCV lymphomas following 24-h treatment that increased over time (FIG. 2B). However, oxamflatin did not mediate any substantial change in MOMP in any of the Eμ-myc lymphomas that overexpress Bcl-2. In contrast and consistent with the data shown in FIG. 2A, romidepsin induced MOMP in 4242Eμ-myc/Bcl-2 and 229Eμ-myc/Bcl-2 and this effect was greatly attenuated or completely lost in the 226Eμ-myc/Bcl-2 and 102Eμ-myc/Bcl-2 lymphomas.

Next, the cell cycle profiles of Eμ-myc/Bcl-2 lymphomas treated with oxamflatin and romidepsin over 3 days were assessed. Treatment of 226Eμ-myc/Bcl-2 (Table 1) or 102Eμ-myc/Bcl-2 lymphomas with oxamflatin or romidepsin over 3 days resulted in a decrease in the percentage of cells in S phase and increase of cells in G1 (Table 1). Using loss of 2n DNA content (sub-G1) as readout for DNA fragmentation and thus apoptosis, neither oxamflatin nor romidepsin induced substantial cell death even following 3 days of continuous exposure to replenished agent. Similar results were seen when 4242Eμ-myc/Bcl-2 (Table 1) and 229Eμ-myc/Bcl-2 lymphomas were treated with oxamflatin. In contrast, treatment of 4242Eμ-myc/Bcl-2 (Table 1) and 229Eμ-myc/Bcl-2 (data not shown) lymphomas with romidepsin resulted in an increase in the percentage of cells showing DNA fragmentation indicative of apoptosis. Taken together, these data show that overexpression of Bcl-2 robustly inhibits the apoptotic activities of the hydroxamate-based HDACi oxamflatin. In contrast, two of the Eμ-myc/Bcl-2 lymphomas that were completely resistant to oxamflatin induced apoptosis were sensitive to romidepsin-mediated apoptosis following >24-h exposure to drug.

To ensure that romidepsin and oxamflatin induced equivalent histone hyperacetylation at doses of each compound that could kill Eμ-myc lymphomas, Western blot analysis was performed to assess the acetylation status of histones H3 and H4. As shown in FIG. 2C, treatment of 4242Eμ-myc lymphomas with 3.0 nmol/L romidepsin and 0.1 mmol/L oxamflatin induced equivalent acetylation of histones H3 and H4 over a 24-h time course. Moreover, addition of 3.0 nmol/L romidepsin to 4242Eμ-myc/Bcl-2 and 226Eμ-myc/Bcl-2 lymphomas resulted in an equivalent increase in histone acetylation in a time-dependent manner. These data indicate that the differential sensitivity of 4242Eμ-myc/Bcl-2 and 226Eμ-myc/Bcl-2 to romidepsin is not related to variations in HDAC inhibitory activity of the compound in lymphomas that are relatively resistant or sensitive to romidepsin-induced apoptosis.

TABLE 1
Cell Cycle analysis of Eμ-myc/Bcl-2 cells treated with HDACi.

| | | | |
|---|---|---|---|
| % subG1 | % G1 | % S phase | % G2/M |

4242/Bcl-2

| | | | | |
|---|---|---|---|---|
| Vehicle 24 hr | 15.7 | 40.5 | 21.4 | 23.9 |
| Vehicle 48 hr | 12.6 | 47.0 | 12.1 | 25.1 |
| Vehicle 72 hr | 25.0 | 41.7 | 9.1 | 21.8 |
| Oxamflatin 24 hr | 8.4 | 59.4 | 8.2 | 18.0 |
| Oxamflatin 48 hr | 5.9 | 62.3 | 8.4 | 15.3 |
| Oxamflatin 72 hr | 4.6 | 66.7 | 9.7 | 15.8 |
| Romidepsin 24 hr | 19.3 | 66.3 | 2.1 | 11.4 |
| Romidepsin 48 hr | 27.2 | 53.2 | 4.6 | 8.5 |
| Romidepsin 72 hr | 55.3 | 34.8 | 3.0 | 7.1 |

226/Bcl-2

| | | | | |
|---|---|---|---|---|
| Vehicle 24 hr | 2.6 | 46.0 | 27.9 | 22.5 |
| Vehicle 48 hr | 1.3 | 55.8 | 17.1 | 24.1 |
| Vehicle 72 hr | 1.4 | 65.7 | 9.8 | 21.4 |
| Oxamflatin 24 hr | 2.7 | 50.0 | 14.4 | 28.8 |
| Oxamflatin 48 hr | 2.6 | 62.6 | 10.8 | 21.4 |
| Oxamflatin 72 hr | 6.5 | 78.2 | 2.5 | 12.0 |
| Romidepsin 24 hr | 3.7 | 48.6 | 16.4 | 26.6 |
| Romidepsin 48 hr | 3.7 | 52.3 | 13.5 | 27.1 |
| Romidepsin 72 hr | 7.8 | 78.3 | 2.5 | 8.7 |

### Example 2

### Apoptotic and Therapeutic Activity of Romidepsin Against Eμ-myc and Eμ-myc/Bcl-2 Lymphomas In Vivo

In vitro data indicated that romidepsin was capable of rapidly killing Eμ-myc lymphomas and could kill 229Eμmyc/Bcl-2 and 4242Eμ-myc/Bcl-2 lymphomas over time but could not kill 226Eμ-myc/Bcl-2 or 102Eμ-myc/Bcl-2 lymphomas. To determine if similar results were observed in vivo, apoptosis assays were performed that involved treatment of lymphoma-bearing mice in vivo with romidepsin, harvesting of tumors over time, and assessment of apoptosis using fluorescence-activated cell sorting-based assays.

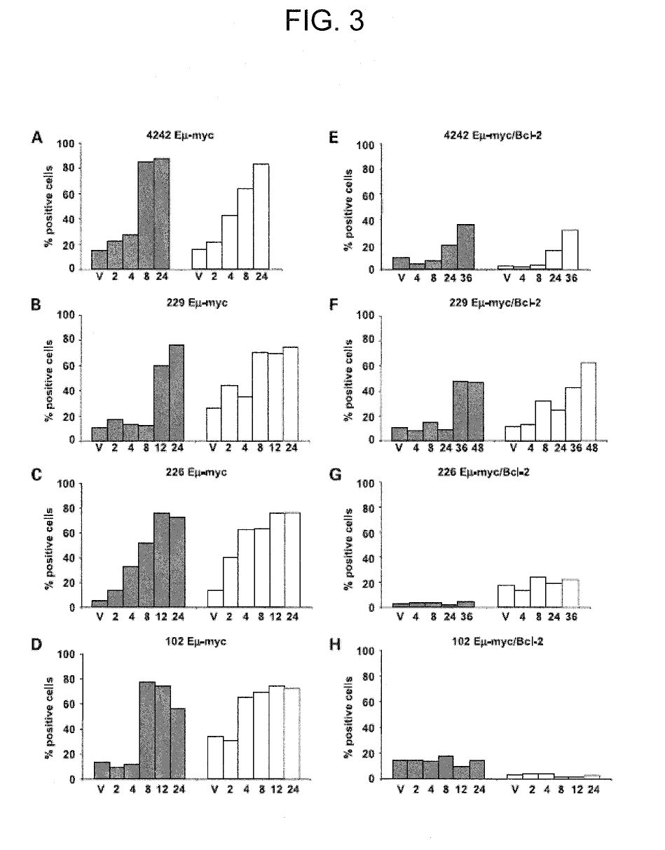

All four Eμ-myc lymphomas grown in the lymph nodes of C57BL/6 mice were sensitive to romidepsin with an increase in apoptotic cells over background detected at 8 to 12 h following addition of romidepsin (FIG. 3A-D). The percentage of apoptosis increased over the 24-h time course using readouts for outer cell membrane damage and DNA fragmentation (FIG. 3A-D). Consistent with the results seen in vitro, all four Eμ-myc/Bcl-2 lymphomas were resistant to romidepsin-induced apoptosis 24 h after exposure to the HDACi (FIG. 3E-H). The 226Eμ-myc/Bcl-2 and 102Eμ-myc/Bcl-2 lymphomas remained insensitive to romidepsin induced apoptosis in vivo, even at the 36 and 48 h time points, respectively (FIGS. 3G and H). However, consistent with in vitro data, 4242Eμ-myc/Bcl-2 (FIG. 3E) and 229Eμ-myc/Bcl-2 (FIG. 3F) lymphomas did undergo apoptosis at later time points following exposure to romidepsin, although as with the in vitro assays the level of apoptosis achieved in these Bcl-2-overexpressing lymphomas at most time points was substantially less than that observed in the parental Eμ-myc lymphomas.

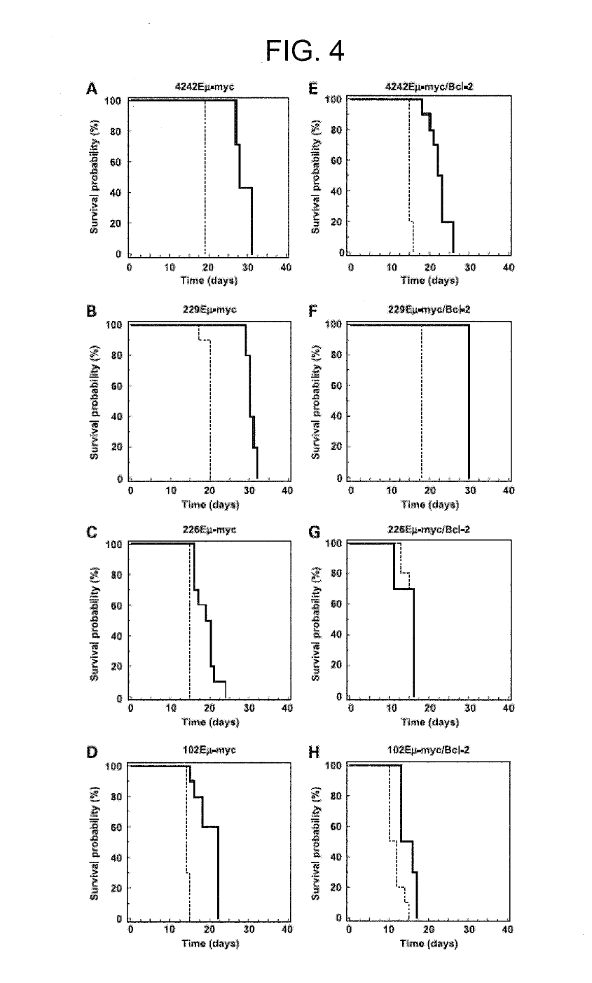

Next, the therapeutic effects of romidepsin against Eμ-myc and Eμ-myc/Bcl-2 lymphomas were assessed to determine if the induction of apoptosis by romidepsin in vivo translated into a therapeutic benefit. For therapy experiments, Eμ-myc lymphomas were transplanted into C57BL/6 mice and treatment with romidepsin or vehicle commenced when WBC counts in the peripheral blood reached a pathologic threshold (>13×10³/μL). The survival of mice bearing Eμ-myc lymphomas treated with romidepsin was significantly extended compared with vehicle-treated mice (FIG. 4A-D). Interestingly, romidepsin also significantly extended the survival of mice bearing 229Eμ-myc/Bcl-2 and 4242Eμ-myc/Bcl-2 lymphomas but provided little or no therapeutic benefit in mice bearing 102Eμ-myc/Bcl-2 or 226Eμ-myc/Bcl-2 lymphomas (FIG. 4E-H).

### Example 3

### Enhanced Expression of Bcl-XL in 226Eρ-myc/Bcl-2 and 102Eμ-myc/Bcl-2 Lymphomas Correlates with Resistance to Romidepsin-Induced Apoptosis

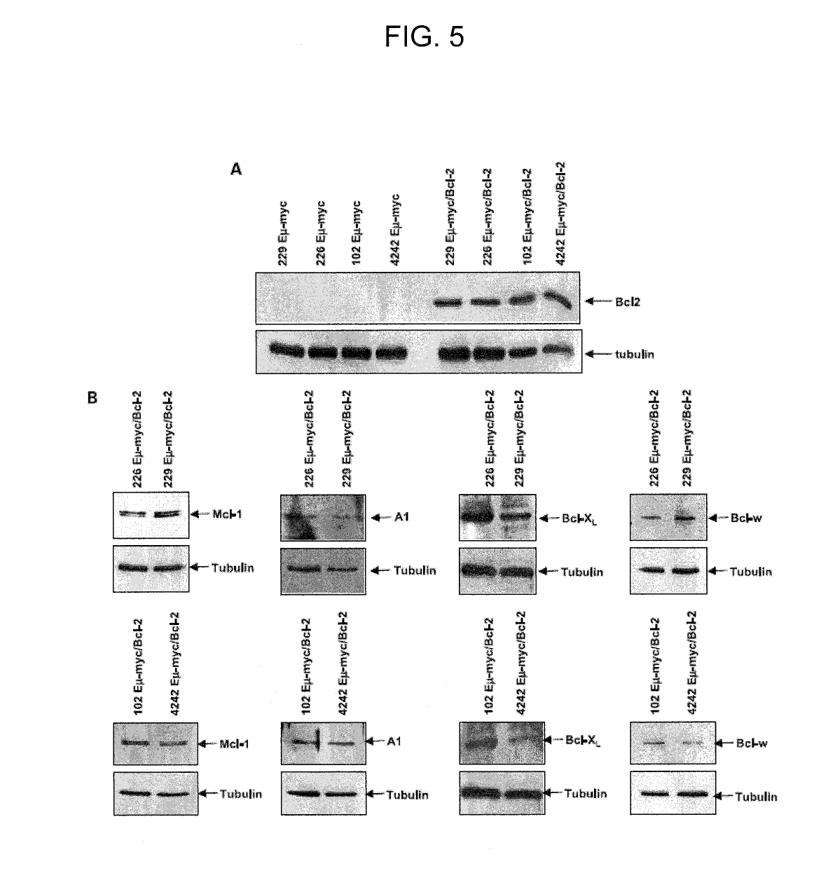

To determine why 226Eμ-myc/Bcl-2 and 102Eμ-myc/Bcl-2 lymphomas remain resistant to romidepsin-induced apoptosis compared with 229Eμ-myc/Bcl-2 and 4242Eμmyc/Bcl-2 lymphomas, expression of prosurvival Bcl-2 proteins in the cells was examined. All cells overexpressed approximately equivalent amounts of exogenous Bcl-2 (FIG. 5A). Next, endogenous expression of prosurvival Bcl-2 family members in these lymphomas was assessed (FIG. 5B). The expression of Bcl-w, Mcl-1, and A1 was approximately equivalent in all Eμ-myc/Bcl-2 lymphomas. In contrast, the levels of Bcl-X_{L} were significantly higher in 226Eμ-myc/Bcl-2 and 102Eμ-myc/Bcl-2 lymphomas compared with 229Eμ-myc/Bcl-2 and 4242Eμmyc/Bcl-2 lymphomas.

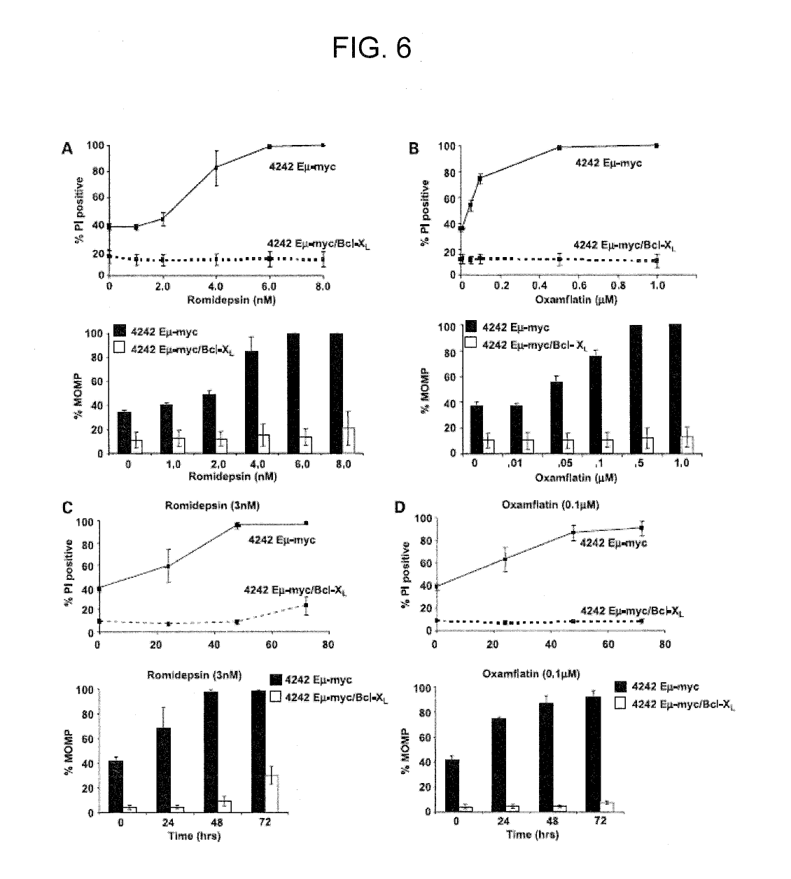

To determine if increased expression of Bcl-X_{L} could confer resistance to romidepsin, 4242Eμ-myc/Bcl-X_{L} lymphomas were produced and tested for sensitivity to HDACi. Treatment of 4242Eμ-myc and 4242Eμ-myc/Bcl-X_{L} lymphomas with increasing concentrations of romidepsin or oxamflatin over 24 h resulted in dose-dependent loss of plasma membrane integrity and mitochondrial function in 4242Eμ-myc lymphomas, whereas 4242Eμ-myc/Bcl-X_{L} lymphomas were unaffected (FIGS. 6A and B). Moreover, cell cycle analysis revealed that DNA fragmentation occurred in Eμ-myc lymphomas in response to increasing doses of oxamflatin and romidepsin, whereas Eμ-myc/Bcl-X_{L} lymphomas arrested in the G1 phase of the cell cycle. Similar results were seen using 102Eμ-myc/Bcl-X_{L} and 229Eμ-myc/Bcl-X_{L} lymphomas. Treatment of 4242Eμ-myc/Bcl-X_{L}, lymphomas with romidepsin or oxamflatin over a 72-h time course resulted in little or no outer cell membrane permeabilization nor any significant decrease in mitochondrial membrane potential (FIGS. 6C and D). In contrast, parental Eμ-myc lymphomas were effectively killed by romidepsin and oxamflatin within the first 24 h (FIGS. 6C and D). Similar results were observed using 102Eμ-myc/Bcl-X_{L} and 229Eμ-myc/Bcl-X_{L} lymphomas.

### Example 4

### Materials and Methods

Eμ-myc Lymphomas, Cell Culture, and Reagents

Eμ-myc, Eμ-myc/Bcl-2, and Eμ-myc/Bcl-X_{L} lymphomas were developed as described previously (Lindemann et al., Proc. Nat. Acad. Sci. USA 104:8071-8078, 2007) and cultured in six-well plates (Greiner Bio-One) in high-glucose DMEM supplemented with 10% FCS, penicillin/streptomycin, 0.1 mmol/L L-asparagine, and 50 μmol/L 2-mercaptoethanol. HDACi were dissolved in DMSO for the preparation of stock solutions (10 mmol/L).

Western Blot Analysis

Eμ-myc lymphoma cells were lysed in lysis buffer [0.15 mol/L NaCl, 10 mmol/L Tris-HCl (pH 7.4), 5 μmol/L EDTA, 1% Triton X-100] supplemented with protease inhibitors (leupeptin, pepstatin, and phenylmethylsulfonyl fluoride; Sigma-Aldrich) as described previously (Lindemann et al., Proc. Nat. Acad. Sci. USA 104:8071-8078, 2007). Proteins (30-50 μg) were separated on 10% or 15% SDS polyacrylamide gels electroblotted onto Immobilon-P nylon membranes (Millipore). Membranes were incubated with the following antibodies: anti-mouse Bcl-2 (BD PharMingen), anti-mouse Bcl-X_{L} (BD PharMingen), anti-mouse Bcl-w (Chemicon Australia), anti-mouse Mcl-1 (Rockland), anti-mouse A1 (Sapphire Biosciences), anti-Flag tag (Sigma-Aldrich), anti-acetylated histone H3 and antiacetylated histone H4 (Upstate Biosystems), anti-β actin (Sigma-Aldrich), and anti-tubulin (Sigma-Aldrich) overnight at 4° C. followed by subsequent incubation with horseradish peroxidase-conjugated secondary antibodies (DAKO) Immunoreactive bands were visualized by enhanced chemiluminescence (Amersham).

In Vitro Cell Death Analysis

Eμ-myc lymphoma cells (5×10⁵/mL) were incubated in the presence of the indicated compounds for 20 h in 1 mL cell culture medium in 24-well plates (Greiner Bio-One). Viability of cells as measured by trypan blue exclusion assay, propidium iodide uptake, Annexin V staining, cell cycle analysis, or tetramethylrhodamine ethyl ester staining were done as described (Lindemann et al., Proc. Nat. Acad. Sci. USA 104:8071-8078, 2007).

Mice

C57BL/6 mice (6-8 weeks old) were used for in vivo apoptosis assays and therapy studies. PCR-based genotyping and Western blotting analysis were used to validate lymphoma genotypes (data not shown).

In Vivo Apoptosis and Therapy Assays

For in vivo apoptosis assays, C57BL/6 mice were injected with Eμ-myc lymphomas (5×10⁵ cells per animal) and after 10 to 15 days on which lymph nodes became well-palpable romidepsin (5.6 mg/kg) was administered i.v. After the indicated time points, mice were sacrificed and cells were harvested from brachial lymph nodes for fluorescence-activated cell sorting-based assays to measure apoptotic signaling (Lindemann et al., Proc. Nat. Acad. Sci. USA 104:8071-8078, 2007). To assess therapeutic efficacy of romidepsin, C57BL/6 mice were injected with Eμ-myc lymphomas of the indicated genotypes i.v. (5×10⁵ cells per animal). Peripheral WBC counts were then monitored until they exceeded 13×10³/μL (Sysmex Hematology Analyzer K-1000) and romidepsin was administered at 5.6 mg/kg i.v. once every 4 days for a total of four injections. Previously, it had been determined that this regimen represented the maximum tolerated dose in lymphoma-bearing mice. Mice in the control cohort received the corresponding amount of vehicle. Cohorts consisted of 8 to 11 mice each, 2 to 3 independently derived lymphomas per genotype. Peripheral WBC counts and body weights were recorded weekly. On signs of major distress or when lymphomas were relapsing as indicated by enlarged brachioaxial lymph nodes, mice were euthanized and a necropsy was done. For analysis of therapeutic efficacy, tumor-induced mortality “events” were recorded. Kaplan-Meier analysis was done and comparisons made using the log-rank (Mantel-Cox) test (MedCalc software version 8.0.2.0).

### Equivalents and Scope

The foregoing has been a description of certain non-limiting preferred embodiments of the invention. Those skilled in the art will recognize, or be able to ascertain using no more than routine experimentation, many equivalents to the specific embodiments of the invention described herein. Those of ordinary skill in the art will appreciate that various changes and modifications to this description may be made without departing from the spirit or scope of the present invention, as defined in the following claims.

In the claims articles such as “a”, “an”, and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise. In addition, the invention encompasses compositions made according to any of the methods for preparing compositions disclosed herein.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Any embodiment, element, feature, application, or aspect of the compositions and/or methods of the invention can be excluded from any one or more claims. For example, in certain embodiments of the invention the biologically active agent is not an anti-proliferative agent. For purposes of brevity, all of the embodiments in which one or more elements, features, purposes, or aspects is excluded are not set forth explicitly herein.

1. A method of treating a lymphoma in a subject, the method comprising the steps of:
a) providing a subject identified as having a lymphoma that expresses Bcl-2; and
b) administering a therapeutically effective amount of romidepsin to the subject.

2. The method of claim 1, wherein cells of the lymphoma overexpress Bcl-2.

3. The method of claim 1, wherein the method comprises determining Bcl-2 expression in the lymphoma cells, wherein Bcl-2 polypeptide expression or Bcl-2 mRNA expression is determined.

4. The method of claim 3, wherein Bcl-2 expression is determined in vitro in a sample from the lymphoma.

5-6. (canceled)

7. The method of claim 1, wherein the lymphoma does not overexpress Bcl-XL.

8. The method of claim 7, wherein expression of Bcl-2 is equal to or greater than expression of Bcl-XL in cells of the lymphoma.

9. The method of claim 7, wherein the lymphoma cells do not express Bcl-XL.

10. The method of claim 1, wherein the method comprises determining Bcl-XL expression in cells of the lymphoma, wherein Bcl-XL polypeptide expression or Bcl-XL mRNA expression is determined.

11. The method of claim 10, wherein Bcl-XL expression is determined in vitro in a sample from the lymphoma.

12-13. (canceled)

14. The method of claim 1, wherein the lymphoma cells do not overexpress P-glycoprotein.

15. The method of claim 1, wherein the method comprises determining P-glycoprotein expression in cells of the lymphoma.

16. The method of claim 1, wherein the lymphoma is a T cell lymphoma selected from the group consisting of a cutaneous T cell lymphoma (CTCL) and peripheral T cell lymphoma (PTCL).

17-18. (canceled)

19. The method of claim 1, wherein the lymphoma is selected from the group consisting of a non-Hodgkin's lymphoma, a Hodgkin's lymphoma, a follicular lymphoma, a B cell lymphoma, a diffuse large B cell lymphoma, a mantle cell lymphoma, and a Burkitt's lymphoma.

20-25. (canceled)

26. The method of claim 1, wherein romidepsin is of the formula:

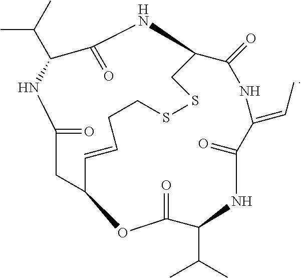

27. The method of claim 1, wherein the lymphoma is selected from the group consisting of a refractory lymphoma, a relapsed lymphoma, and a steroid-resistant lymphoma.

28-29. (canceled)

30. The method of claim 1, wherein the therapeutically effective amount of romidepsin ranges from approximately 0.5 mg/m² to approximately 28 mg/m².

31-38. (canceled)

39. The method of claim 1, wherein romidepsin is administered intravenously.

40. The method of claim 1, wherein romidepsin is administered bimonthly, monthly, triweekly, biweekly, weekly, twice a week, daily, or at variable intervals.

41. The method of claim 1, wherein romidepsin is administered weekly.

42. The method of claim 1, further comprising administering a compound selected from the group consisting of a second anti-neoplastic agent, an inhibitor of Bcl-XL expression or activity, a cytotoxic agent, a steroidal agent, a proteasome inhibitor, and a kinase inhibitor.

43-45. (canceled)

46. The method of claim 42, wherein the steroidal agent is selected from the group consisting of alclometasone diproprionate, amcinonide, beclomethasone diproprionate, betamethasone, betamethasone benzoate, betamethasone diproprionate, betamethasone sodium phosphate, betamethasone sodium phosphate and acetate, betamethasone valerate, clobetasol proprionate, clocortolone pivalate, cortisol (hydrocortisone), cortisol (hydrocortisone) acetate, cortisol (hydrocortisone) butyrate, cortisol (hydrocortisone) cypionate, cortisol (hydrocortisone) sodium phosphate, cortisol (hydrocortisone) sodium succinate, cortisol (hydrocortisone) valerate, cortisone acetate, desonide, desoximetasone, dexamethasone, dexamethasone acetate, dexamethasone sodium phosphate, diflorasone diacetate, fludrocortisone acetate, flunisolide, fluocinolone acetonide, fluocinonide, fluorometholone, flurandrenolide, halcinonide, medrysone, methylprednisolone, methylprednisolone acetate, methylprednisolone sodium succinate, mometasone furoate, paramethasone acetate, prednisolone, prednisolone acetate, prednisolone sodium phosphate, prednisolone tebutate, prednisone, triamcinolone, triamcinolone acetonide, triamcinolone diacetate, and triamcinolone hexacetonide or a synthetic analog thereof, or a combination thereof.

47-49. (canceled)

50. The method of claim 42, wherein the proteasome inhibitor is selected from the group consisting of bortezomib (VELCADE®), peptide boronates, salinosporamide A (NPI-0052), lactacystin, epoxomicin (Ac(Me)-Ile-Ile-T′hieu-EX), MG-132 (Z-Leu-Leu-Leu-al), PR-171, PS-519, eponemycin, aclacinomycin A, CEP-1612, CVT-63417, PS-341-(pyrazylcarbonyl-Phe-Leu-boronate), PSI (Z-Ile-Glu(OtBu)-Ala-Leu-al), MG-262 (Z-Leu-Leu-Leu-bor), PS-273 (MNLB), omuralide (clasto-lactacystin-p-lactone), NLVS (Nip-Leu-Leu-Leu-vinyl sulfone), YLVS (Tyr-Leu-Leu-Leu-vs), dihydroeponemycin, DFLB (dansyl-Phe-Leu-boronate), ALLN (Ac-Leu-Leu-Nle-al), 3,4-dichloroisocoumarin, 4-(2-aminoethyl)-benzenesulfonyl fluoride, TMC-95A, gliotoxin, EGCG ((−)-epigallocatechin-3-gallate), and YU101 (Ac-hFLFL-ex).

51. (canceled)

52. The method of claim 42, wherein the second anti-neoplastic agent is administered prior to, simultaneously with or following the administration of romidepsin.

53. (canceled)

54. A method of treating Bcl-2 expressing lymphoma cells, the method comprising the steps of:
a) providing lymphoma cells identified as expressing Bcl-2; and
b) administering romidepsin to the cells.

55. The method of claim 54, wherein romidepsin is administered to the cells at a concentration and for a period of time sufficient to kill the cells.

56. The method of claim 54, wherein the cells overexpress Bcl-2.

57. The method of claim 54, wherein the method comprises determining Bcl-2 expression in the cells, prior to the step of administering, wherein Bcl-2 polypeptide expression or Bcl-2 mRNA expression is determined.

58-59. (canceled)

60. The method of claim 54, wherein the cells do not overexpress Bcl-XL.

61. The method of claim 60, wherein expression of Bcl-2 is equal to or greater than expression of BCl-XL in the cells.

62. The method of claim 60, wherein expression of Bcl-2 is at least twice the expression of Bcl-XL in the cells.

63. The method of claim 54, wherein the method comprises determining Bcl-XL expression in the cells, wherein Bcl-XL polypeptide expression or Bcl-XL mRNA expression is determined.

64-65. (canceled)

66. The method of claim 54, wherein romidepsin is administered for a period of about 24 hours to about 72 hours.

67. (canceled)

68. The method of claim 54, wherein romdepsin is administered at a concentration of about 1 nmol/L to about 3 nmol/L.

69. (canceled)

70. A method for identifying a candidate for treatment with romidepsin, the method comprising the steps of:
a) providing a sample from a subject having a lymphoma; and
b) determining Bcl-2 expression in cells of the lymphoma, wherein expression of Bcl-2 in cells of the lymphoma indicates that the subject is a candidate for treatment with romidepsin.

71. The method of claim 70, further comprising
determining and Bcl-XL expression in cells of the lymphoma, wherein the expression of Bcl-2 which is equal to or greater than the expression of Bcl-XL in cells of the lymphoma indicates that the subject is a candidate lymphoma patient for treatment with romidepsin.

72. A method for identifying a candidate lymphoma patient for treatment with romidepsin, the method comprising the steps of:
a) providing a sample from a subject having a lymphoma, and
b) determining Bcl-XL expression in cells of the lymphoma, wherein a lack of overexpression of Bcl-XL in cells of the lymphoma indicates that the subject is a candidate lymphoma patient for treatment with romidepsin.

73. A method of treating a lymphoma in a subject, the method comprising the steps of:
a) providing a subject identified as having a lymphoma that lacks expression of Bcl-XL; and
b) administering a therapeutically effective amount of romidepsin to the subject.
